(19) United States
(12) Patent Application Publication
(10) Pub. No.: US 2026/0090975 A1
(43) Pub. Date: Apr. 2, 2026

(54) COSMETIC COMPOSITION, COMPRISING UV FILTER COMPOUND AND CROSSLINKED POLYESTER COMPONENT

(71) Applicant: Momentive Performance Materials Inc., Niskayuna, NY (US)

(72) Inventors: Sarah Peeler Black, New York, NY (US); Benjamin FALK, Yorktown Heights, NY (US); Ashitha Kandikkal, Bangalore (IN); Yan Ling Lee, Elmwood Park, NJ (US); Amar Pawar, Briarcliff Manor, NY (US); Xu Qin, Northvale, NJ (US); Polina Tsupko, Bronx, NY (US); Gianna Villani, Bronx, NY (US)

(21) Appl. No.: 19/342,157

(22) Filed: Sep. 26, 2025

(30) Foreign Application Priority Data
Sep. 27, 2024 (IN) 202411073259

(51) Int. Cl. A61K 8/85; A61K 8/04; A61Q 17/04

(52) U.S. Cl. CPC A61K 8/85; A61K 8/042; A61Q 17/04; A61K 2800/26; A61K 2800/805

(57) ABSTRACT

The present invention provides a cosmetic composition, comprising at least one UV filter compound (A), at least one crosslinked polyester component (B), optionally at least one cosmetically acceptable carrier (C), and optionally one or more other components commonly used in the cosmetic field (D).

The present invention provides a cosmetic composition, comprising at least one UV filter compound (A), at least one crosslinked polyester component (B), optionally at least one cosmetically acceptable carrier (C), and optionally one or more other components commonly used in the cosmetic field (D).

### STATEMENT OF THE PROBLEM

Crosslinked polymers are added to manipulate the sensory, texture, rheology, and optical performance of a variety of cosmetic products. Traditional silicone elastomers have limited versatility in terms of compatibility with certain ingredients, including chemical UV filters. Therefore, although the performance of silicone elastomers is unparalleled, there is a demand for alternatives to silicone elastomers for use in sunscreens and other personal care formulations.

### STATE OF THE ART

The personal care industry thrives on being able to deliver multiple performance products based on mixtures of several components, with each having performance characteristics important to or desirable in the final formulation. Silicone gels are commonly added in a variety of personal care formulations to enhance their aesthetics with respect to sensory, texture, rheology, and optical performance. See for example, U.S. Pat. Nos. 4,987,169; 5,654,362; 5,760,116; 6,423,322; and 5,811,487.

Crosslinked polymers are added to manipulate the sensory, texture, rheology, and optical performance of a variety of cosmetic products. Silicone elastomers are particularly important because they can form elastic particles of three-dimensional polymeric dimethicone and provide a beneficial sensory, texture, and optical effect to cosmetic products. However, traditional silicone elastomers have limited versatility in terms of compatibility with polar solvents or emollients such as hydrocarbon oils, plant-based oils, glycerin, and water. Therefore, although the performance of silicone elastomers is unparalleled, there is a demand for alternatives to silicone elastomers. In particular, there is a demand in the marketplace for non-silicone based elastomer materials.

US20210059924A1 disclosed a polyurethane elastomeric rubber composition containing a bio-based polyol cross-linked with a bio-based isocyanate. The cross-linked polyurethane elastomer rubber is in further aspect of the invention included in a gel after being milled in the presence of a bio-based emollient or mixture of bio-based emollients. The polyurethane elastomeric gel has good compatibility with cosmetic and natural oils and can be used as a gelling agent for these oils among other desirable cosmetic formulary roles.

Polyesters are a class of compounds that contain an ester functional group in their polymer chain. The ester group can be hydrolyzed when treated with certain biological catalysts or certain mixed cultures of microorganisms which renders a large number of polyesters biodegradable. There is a growing interest in recent years to design and develop biobased polyesters from renewable resources as emollients, emulsifiers, film formers, or other functional ingredients for personal care applications. See for example, U.S. Pat. Nos. 8,414,906; 9,334,358; 6,540,987; and 7,820,758. However, no polyester elastomer or polyester elastomer gel has yet been reported that provides multiple benefits to consumers as a substitute to silicone gel.

### DETAILED DESCRIPTION OF THE INVENTION

The present invention provides a cosmetic composition, comprising:
(A) at least one UV filter compound,
(B) at least one crosslinked polyester component, comprising a crosslinked polyester which is reaction product of:
(i) at least one compound selected from a polycarboxylic acid, a polycarboxylic acid ester, and combinations thereof,
(ii) at least one polyol, and
(iii) optionally one or more monofunctional component selected from
(a) a monofunctional carboxylic acid; and
(b) a monofunctional alcohol,
(C) optionally at least one cosmetically acceptable carrier, and
(D) optionally one or more other components commonly used in the cosmetic field, different from components (A), (B) or (C).

As will be described in more detail below the crosslinked polyester component (B) (sometimes called polyester elastomer) can be used as dry powder or as a gel which is obtained by swelling the crosslinked polyester e.g. under shear force in a solvent (such as a low molecular weight emollient) to form a uniform polyester gel or paste having a wide viscosity range. The gel is usually a semi-solid colloid formed by suspending the crosslinked polyester in the solvent. The solid particles of the crosslinked polyester in the gel are supposed to form a three-dimensional network that captures and holds the solvent. The crosslinked polyester gel is usually a stable, jelly-like substance that is soft and flexible. These crosslinked polyester components (B) in particular provided as gels in a solvent are expected to deliver superior performance benefits such as improved sensory, structuring, and rheological performance and improved compatibility with the one UV filter compounds (A) to the cosmetic compositions compared to other elastomers disclosed previously.

The crosslinked polyester component (B) (polyester elastomer) comprises suitably the reaction product of:
at least one poly-carboxylic acid, at least one poly-carboxylic acid
ester, or a combination thereof; and
at least one polyol; and/or
at least one monofunctional-carboxylic acid; and/or
at least one monofunctional-alcohol.

In an aspect, the crosslinked polyester component (B) is a reaction product of:
compound (i) selected from the group consisting of
(1) one or more poly-carboxylic acids of formula (I)

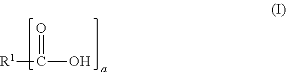

wherein
R¹ is selected from the group consisting of C2-C52 alkyl group, C2-C52 heteroalkyl group, C2-C52 alkene group, C2-C52 heteroalkene group, C3-C52 cyclic group, or C2-C52 heterocyclic group, and
a is an integer from 2 to 10, or
(2) one or more carboxylic acid esters of formula (II)

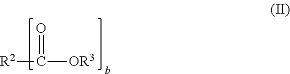

wherein
R² is selected from the group consisting of C2-C200 alkyl group, C2-C200 heteroalkyl group, C2-C200 alkene group, C2-C200 heteroalkene group, C2-C200 alkyne group, C2-C200 heteroalkyne group, C2-C200 cyclic group, or C2-C200 heterocyclic group; and
R³ is selected from the group consisting of C1-C22 alkyl group, C2-C22 alkylene group, or C3-C22 cyclic group, and
b is an integer from 2 to 10, and
(3) a combination (1) and (2) thereof,
compound (ii) selected from one or more polyols of formula (III)

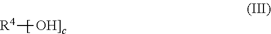

wherein
R⁴ is C2-C200 alkyl group, C2-C200 heteroalkyl group, C2-C200 alkene group, C2-C200 heteroalkene group, C2-C200 alkyne group, C2-C200 heteroalkyne group, C3-C200 cyclic group, or C2-C200 heterocyclic group; and
c is an integer from 2 to 10, and
optional compound (iii) selected from the group consisting of
(a) one or more mono-carboxylic acids of formula (IV)

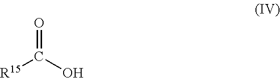

wherein
R⁵ is C2-C52 alkyl group, C2-C52 heteroalkyl group, C2-C52 alkene group, C2-C52 heteroalkene group, C3-C52 cyclic group, or C2-C52 heterocyclic group,
(b) one or more mono-alcohols of formula (V)

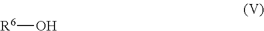

wherein
R⁶ is C2-C52 alkyl group, C2-C52 heteroalkyl group, C2-C52 alkene group, C2-C52 heteroalkene group, C3-C52 cyclic group, or C2-C52 heterocyclic group.

In an aspect, the method of preparing a polyester elastomer (B) comprising reacting:
(i) at least one poly-carboxylic acid, at least one poly-carboxylic acid ester, or a combination thereof; and
(ii) at least one polyol; and/or
(iii) at least one mono-carboxylic acid; and/or
(iv) at least one mono-alcohol.

In an aspect, the crosslinked polyester elastomer (B) is the reaction product of:
(i) at least one polycarboxylic acid, at least one polycarboxylic acid ester, or a combination thereof; and
(ii) at least one polyol; and/or
(iii) at least one mono-carboxylic acid; and/or
(iv) at least one mono-alcohol.

In an aspect, the crosslinked polyester component (B) is reaction product of:
(i) one or more poly-carboxylic acids selected from di-carboxylic acids, tri-carboxylic acids and combinations thereof;
(ii) one or more polyols selected from diols, triols and combinations thereof, and
(iii) optionally one or more monofunctional carboxylic acids.

In an aspect, the crosslinked polyester (B) is reaction product of:
(i) one or more dicarboxylic acids
(ii) one or more triols, and
(iii) optionally one or more monofunctional carboxylic acids.

In an aspect, the preparation of crosslinked polyester component (B) is without solvent or emollient. In an aspect, the preparation of crosslinked polyester component (B) is with solvent or emollient as defined herein.

In an aspect, crosslinked polyester component (B) is only comprised of polyester. In an aspect, polyester elastomer is only comprised of crosslinked polyester. In an aspect, crosslinked polyester component (B) is comprised of crosslinked polyester and non-crosslinked polyester.

In an aspect, crosslinked polyester component (B) is comprised of polyester and solvent or emollient. In an aspect, crosslinked polyester component (B) is comprised of crosslinked polyester and solvent or emollient. In an aspect, the crosslinked polyester component (B) is comprised of crosslinked polyester, non-crosslinked polyester, and solvent or emollient.

In an aspect, the crosslinked polyester component (B) is a powder.

The crosslinked polyester component (B) form crosslinked polymer networks. As is well-known to a skilled person in the art such crosslinked polymer are not (completely) soluble and certain methods are available to characterize them, including for example sol-gel analysis determination of the gel fraction), swelling ratio analysis (determination of the swelling ratio), and mechanical analysis (e.g. determination of the modulus) (see e.g. Polym. Chem., 2024, 15, 219-247).

In an aspect, the fraction of the crosslinked polyester component (B) which is not soluble in ethyl acetate (gel fraction) is greater than or equal to 20%. In an aspect, the fraction of polyester elastomer which is not soluble in ethyl acetate (gel fraction) is greater than or equal to 40%. In an aspect, the fraction of the polyester elastomer which is not soluble in ethyl acetate (gel fraction) is greater than or equal to 50%. In an aspect, the fraction of the polyester elastomer which is not soluble in ethyl acetate (gel fraction) is greater than or equal to 60%. In an aspect, the fraction of the polyester elastomer which is not soluble in ethyl acetate (gel fraction) is greater than or equal to 70%. The gel fraction is suitably defined as

$$Gel fraction ( \% ) = 100 \times \frac{weight of dried gel ( insoluble residue of the extraction )}{total weight of the polyester elastomer used in the extraction} .$$

In an aspect, the gel fraction can be determined with an extraction method such as the Soxhlet extraction described herein.

In an aspect, crosslinked polyester component (B) is comprised of polyester elastomer and solvent or emollient as defined herein. In an aspect, polyester elastomer composition is comprised of polyester elastomer without solvent or emollient as defined herein. In an aspect, crosslinked polyester component (B) is a gel or a powder.

In an aspect, the crosslinked polyester component (B) is a polyester elastomer combined with one or more solvents or emollients, which can be converted to a polyester elastomer gel (the swollen polyester elastomer), for example, by applying a shear force to the composition.

### DETAILED DESCRIPTION

### I. Definitions

Unless otherwise indicated, any atom with unsatisfied valences is assumed to have hydrogen atoms sufficient to satisfy the valences.

It is to be noted that the term “a” or “an” entity refers to one or more of that entity; for example, “a nucleic acid sequence,” is understood to represent one or more nucleic acid sequences, unless stated otherwise. As such, the terms “a” (or “an”), “one or more,” and “at least one” can be used interchangeably herein.

Furthermore, “and/or” where used herein, is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

The term “about” is used herein to mean approximately, roughly, around, or in the regions of. When the term “about” is used in conjunction with a numerical range, it modifies that range by extending the boundaries above and below the numerical values set forth. In general, the term “about” can modify a numerical value above and below the stated value by a variance of, e.g., 10 percent, up or down (higher or lower).

As used herein, the following definitions shall apply unless otherwise indicated. For purposes of the present disclosure, the chemical elements are identified in accordance with the Periodic Table of the Elements, CAS version, and the Handbook of Chemistry and Physics, 75th Ed. 1994. Additionally, general principles of organic chemistry are described in “Organic Chemistry,” Thomas Sorrell, University Science Books, Sausalito: 1999, and “March's Advanced Organic Chemistry,” 6th Ed., Smith, M. B. and March, J., eds. John Wiley & Sons, New York: 2007, the entire contents of which are hereby incorporated by reference.

The term “hydrocarbon”, as used herein by itself or as part of a group, refers to a straight- or branched-chain aliphatic series of one to two hundred carbon atoms, i.e., a C1-C200 hydrocarbon, or the number of carbon atoms designated, e.g., a C1 hydrocarbon such as a methyl, a C2 hydrocarbon such as ethyl, etc. In one embodiment, the hydrocarbon is a C2-C200 hydrocarbon group. In an embodiment, the hydrocarbon is a C6-C60 hydrocarbon group. In an embodiment, the hydrocarbon is a C6-C60 hydrocarbon group. In an embodiment, the hydrocarbon is a C2-C60 hydrocarbon group. In an embodiment, the hydrocarbon is a C5-C22 hydrocarbon group. Examples of hydrocarbon groups include butyl, octyl, decyl, lauryl, cetyl (palmityl), and stearyl.

The term “alkyl”, as used herein by itself or as part of a group, refers to a straight or branched-chain aliphatic hydrocarbon containing one to two hundred carbon atoms, i.e., a C2-C200 alkyl, or the number of carbon atoms designated, e.g., a C1 alkyl such as methyl, a C2 alkyl such as ethyl, etc. In one embodiment, the alkyl is a C2-C200 alkyl group. In another embodiment, the alkyl is a C6-C60 alkyl group. In another embodiment, the alkyl is a C2-C60 alkyl group. In another embodiment, the alkyl is a C5-C22 alkyl group. Examples of alkyl group include butyl, octyl, decyl, lauryl, cetyl (palmityl), and stearyl.

The term “alkene”, as used herein by itself or as part of a group, refers to an alkyl group containing one, two, three, or more carbon-to-carbon double bonds. In one embodiment, the alkene group is a C2-C200 alkylene group. In another embodiment, the alkene group is a C6-C60 alkene group. In another embodiment, the alkene group is a C6-C60 alkene group. In another embodiment, the alkene group is a C2-C60 alkene group. In another embodiment, the alkene group is a C5-C22 alkene group.

The term “alkyne”, as used herein by itself or as part of a group, refers to an alkyl group containing one, two, three, or more carbon-to-carbon triple bonds. In another embodiment, the alkyne is a C2-C200 alkyne group.

The term “cyclic”, as used herein by itself or as part of a group, refers to a stable cyclic compound containing three or more atoms. In an embodiment, the cyclic is a C3-C200 cyclic group. In an embodiment, the cyclic is a C6-C60 cyclic group. In an embodiment, the cyclic is a C5-C22 cyclic group. Examples of cyclic compound include benzene, cyclopentane, and cyclohexane.

The term “heteroalkyl”, as used herein by itself or as part of a group, refers to a stable straight or branched chain alkyl radical containing two to two hundred carbon atoms and at least one heteroatom, which can be the same or different, selected from O, N, or S, wherein the sulfur atom(s) can optionally be oxidized. The heteroatoms can be placed at any interior position of the heteroalkyl group or at a position at which the heteroalkyl group is attached to the remainder of the molecule. In an embodiment, the heteroalkyl is a C6-C60 heteroalkyl group. In an embodiment, the heteroalkyl is a C2-C60 heteroalkyl group. Examples of heteroalkyl compound include succinyl, adipoyl, and sebacoyl.

The term “heteroalkene”, as used herein by itself or as part of a group, refers to a stable straight or branched chain alkene radical containing two to two hundred carbon atoms and at least one heteroatom, which can be the same or different, selected from O, N, or S, wherein the sulfur atom(s) can optionally be oxidized. The heteroatoms can be placed at any interior position of the heteroalkyl group or at a position at which the heteroalkyl group is attached to the remainder of the molecule. In an embodiment, the heteroalkene is a C6-C60 heteroalkene group. In an embodiment, the heteroalkene is a C2-C60 heteroalkene group. Examples of heteroalkene compound include oleoyl, ricinolyl, and linoleoyl.

The term “heteroalkyne”, as used herein by itself or as part of a group, refers to a stable straight or branched chain alkyne radical containing two to two hundred carbon atoms and at least one heteroatom, which can be the same or different, selected from O, N, or S, wherein the sulfur atom(s) can optionally be oxidized. The heteroatoms can be placed at any interior position of the heteroalkyl group or at a position at which the heteroalkyl group is attached to the remainder of the molecule. The term “heterocyclic”, as used herein by itself or as part of a group, refers to a stable cyclic compound containing three or more atoms and at least one heteroatom, which can be the same or different, selected from O, N, or S. In an embodiment, the heterocyclic is a C2-C200 heterocyclic group. In an embodiment, the heterocyclic is C6-C60 heterocyclic group.

The term “heterocyclic”, as used herein by itself or as part of a group, refers to a stable cyclic compound containing two or more carbons atoms and at least one heteroatom, which can be the same or different, selected from O, N, or S, wherein the sulfur atom(s) can optionally be oxidized. In an embodiment, the heterocyclic is a C2-C200 heterocyclic group. In an embodiment, the heterocyclic is a C6-C60 heterocyclic group. In an embodiment, the heterocyclic is a C5-C22 heterocyclic group. Examples of heterocyclic compounds include furan, oxolane, and thiophene.

As used herein, the term “olefin” refers to any species having at least one ethylenic double bond such as normal and branched chain aliphatic olefins, cycloaliphatic olefins, aryl substituted olefins, and the like. An olefin can comprise terminal double bond(s) (“terminal olefin”) and/or internal double bond(s) (“internal olefin”) and can be cyclic or acyclic, linear or branched, optionally substituted. The total number of carbon atoms can be from 1 to 100, or from 1 to 40; the double bonds can be unsubstituted or mono-, bi-, tri- or tetrasubstituted.

As used herein, the term “polyolefin” refers to a homopolymer or copolymer of ethylene, propylene, butenes and other unsaturated aliphatic hydrocarbons, vinyl esters (e.g. vinyl acetate), or (meth)acrylics (e.g. butyl acrylate, acrylic acid). Generally, the polyolefin will be a polymer of ethylene, propylene or copolymer thereof, or a copolymer of ethylene or propylene with one or more C4-C12 α-olefin aliphatic comonomers.

A gel is a disperse system comprising at least two components: a solid component and a liquid component. The solid component forms a sponge-like, three-dimensional network whose pores are filled by a liquid. The liquid component is thus immobilized in the solid. In the gel of the invention the solid component is a three-dimensional network formed of the cross-linked polyester elastomer, and the liquid component is formed of one or more solvents or emollients as defined herein. A gel is a semi-solid that can have properties ranging from soft and weak to hard and tough. Gels are also defined as a substantially dilute cross-linked system.

An elastomer gel is thus made from the elastomer powders or particles swelled or dispersed in a liquid, such as a solvent or an emollient, to form a gel. The ability to swell is commonly expressed by the swelling ratio as explained herein.

The term “SPF” refers to sun protection factor, and it measures the degree of sunscreen protection against UV rays.

The term “UV filter” (component (A) refers to a compound that blocks or absorbs ultraviolet (UV) light). UV filters can be physical (Zinc Oxide, Titanium Oxide) or chemical (UVC, UVB, UVA filters).

The term “UVC filter” refers to a UV filter that absorbs UV light in the range of 200-280 nm.

The term “UVB filter” refers to a UV filter that absorbs UV light in the range of 280-320 nm wavelengths. Redness and sunburn are primarily due to UVB exposure.

The term “UVA filter” refers to a UV filter that absorbs UV light in the range of 400 to 320 nm. It is broken up into UVA I (340 to 400 nm) and UVA II (320 to 340 nm).

The term “broad spectrum” refers to a UV filter that absorbs or blocks the entirety of the UV spectrum, including both UVB and UVA rays. Sunscreens can be broad spectrum UV filters if they possess both UVA and UVB chemical filters, or if they are a physical filter, such as the ones listed above.

Cosmetically acceptable carriers (C) can be suitably selected from the group consisting of water, solvents, emollients, fatty acids, fatty alcohols, thickeners and combinations thereof. Preferably they are selected from the solvents and emollients as described herein below.

Various aspects of the disclosure are described in greater detail below.

### DESCRIPTION OF THE FIGURES

FIG. 1 is a line graph showing the particle size distribution (determined as described herein) of the polyester elastomer gel prepared by processing the polyester elastomer of Example 4 with coco-caprylate/caprate solvent or emollient.

FIG. 2 features images of mineral UV filter day creams (formulation such as in Example 21) upon application on the skin (top) and after rub-in (bottom).

FIG. 3 features images of a mineral day cream formulation (such as in Example 21) with a polyester gel (B).

FIG. 4 depicts the stability of emulsion-based sunscreen formulations (such as in Example 23) without any elastomer (blank), with silicone elastomer gel Velvesil DM LC and with a polyester gel (B) after centrifugation at 4500 rpm for 10 minutes.

FIG. 5 depicts stability of an anhydrous sunscreen formulation (such as in Example 19) with silicone elastomer gel Velvesil DM LC and with a polyester gel (B) after storage at 50° C. for 24 hours.

FIG. 6 is a comparison of mineral UV filter dispersion in a mineral UV filter day cream formulation (such as in Example 21) with a polyester gel (B) against a formulation with silicone elastomer gel, Velvesil DM LC.

FIG. 7 is a Hegman gauge of mineral UV filter sunscreen formulations (such as in Example 22) with a polyester gel (B) compared to silicone elastomer gel Velvesil DM LC.

FIG. 8 is SEM imaging showing dispersion of mineral UV filters in formulations (such as in Example 22) with a polyester gel (B) and a silicone elastomer gel Velvesil DM LC.

FIG. 9 is Visioscan imaging of wrinkle reduction of sun gel formulation (such as in Example 19) with silicone elastomer gel Velvesil DM LC and with a polyester gel (B).

FIG. 10 is a visual comparison of sunscreen sticks (such as in Example 25) with polyester gel (B) and silicone elastomer gel Velvesil DM LC.

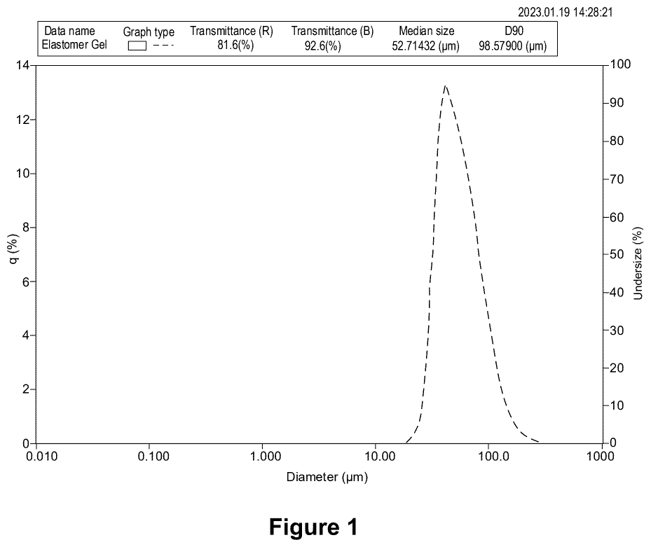

A. Components of the crosslinked polyester component (B)

### 1. Poly-Carboxylic Acids (i)

In some aspects, the at least one poly-carboxylic acid may be a compound of formula (VI)

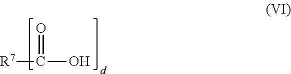

wherein
R7 is C2-C200 alkyl group, C2-C200 heteroalkyl group, C2-C200 alkene group, C2-C200 heteroalkene group, C2-C200 alkyne group, C2-C200 heteroalkyne group, C3-C200 cyclic group, or C2-C200 heterocyclic group; and
d is an integer from 2 to 10.

In a further aspect, the compound is formula (I), wherein R1 is C6-C60 alkyl group, C6-C60 heteroalkyl group, C6-C60 alkene group, C6-C60 heteroalkene group, C6-C60 cyclic group, or C6-C60 heterocyclic group; and a is an integer from 2 to 10. In an aspect, the compound is formula (I), wherein a is 2, 3, 4, 5, 6, 7, 8, 9, or 10. In an aspect, the compound is formula (I), wherein a is an integer from 2 to 6. In another aspect, the compound is formula (I), wherein a is 2, 3, 4, 5, or 6.

In an aspect, the polycarboxylic acid is a product of a compound of formula (I), and/or a compound of formula (III) with a compound of formula (V).

In an aspect, the at least one polycarboxylic acid may be selected from the group consisting of citric acid, isocitric acid, aconitic acid, propane-1,2,3-tricarboxylic acid, trimesic acid, carballylic acid, C54 trimer acid, mellitic acid, and combinations thereof. In a further aspect, the at least one polycarboxylic acid may be selected from the group consisting of citric acid, C54 trimer acid, and combinations thereof.

In an aspect, C36 dimer acid is a dicarboxylic acid prepared by dimerizing unsaturated linoleic fatty acid from plant oil.

In an aspect, C54 trimer acid is a polycarboxylic acid prepared by trimerizing unsaturated fatty acids from plant oil.

In some aspects, the unsaturated fatty acids are palmitoleic acid, oleic acid, elaidic acid, vaccenic acid, linoleic acid, linoelaidic acid, or linolenic acid.

In some aspects, the plant oils are soybean oil, safflower oil, linseed oil, corn oil, sunflower oil, olive oil, canola oil, sesame oil, cottonseed oil, palm oil, rapeseed oil, tung oil, peanut oil, or milkweed oil.

In an aspect, the at least one carboxylic acid may be a dicarboxylic acid. In an aspect, the dicarboxylic acid is a compound of formula (VII)

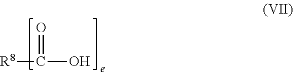

wherein
R⁸ is C2-C200 alkyl group, C2-C200 heteroalkyl group, C2-C200 alkylene group, C2-C200 heteroalkylene group, C2-C200 alkyne group, C2-C200 heteroalkyne group, C3-C200 cyclic group, or C2-C200 heterocyclic group; and
e is 2.

In another aspect, the compound is formula (VII), wherein R⁸ is C2-C200 alkyl group, C2-C200 heteroalkyl group, C2-C200 alkene group, or C2-C200 heteroalkene group and e is 2. In a further aspect, the compound is of formula (VII), wherein R⁸ is C2-C60 alkyl group, C2-C60 heteroalkyl group, C2-C60 alkene group, or C2-C60 heteroalkene group and e is 2. In an aspect, the compound is of formula (VII), wherein R⁸ may be succinyl, adipoyl, sebacoyl, dilinoleyl, or trilinoleyl.

In an aspect, the compound is formula (VII), wherein e may be 1 or 2.

In some aspects, the dicarboxylic acid may be selected from the group consisting of malonic acid, succinic acid, fumaric acid, glutaric acid, adipic acid, pimelic acid, suberic acid, azelaic acid, sebacic acid, undecanedioic acid, dodecanedioic acid, tridecanedioic acid, hexadecanedioic acid, C21 dimer acid, C36 dimer acid, hydrogenated C36 dimer acid, aspartic acid, glutamic acid, tartaric acid, malic acid, and combinations thereof. In a further aspect, the dicarboxylic acid may be selected from the group consisting of malonic acid, succinic acid, adipic acid, azelaic acid, sebacic acid, undecanedioic acid, dodecanedioic acid, C21 dimer acid, C36 dimer acid, hydrogenated C36 dimer acid, and combinations thereof. In one aspect, the dicarboxylic acid is selected from the group consisting of succinic acid, adipic acid, azelaic acid, sebacic acid, undecanedioic acid, dodecanedioic acid, C21 dimer acid, C36 dimer acid, hydrogenated C36 dimer acid, and combinations thereof.

In an aspect, the poly-carboxylic acid is biobased or naturally derived.

### 2. Polycarboxylic Acid Ester (i)

In an aspect, the at least one polycarboxylic acid ester may be a compound of formula (VIII)

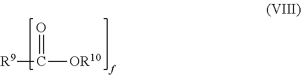

wherein
R⁹ is C1-C22 alkyl group, C2-C22 alkylene group, or C3-C22 cyclic group;
R¹⁰ is C2-C200 alkyl group, C2-C200 heteroalkyl group, C2-C200 alkene group, C2-C200 heteroalkene group, C2-C200 alkyne group, C2-C200 heteroalkyne group, C2-C200 cyclic group, or C2-C200 heterocyclic group; and
f is an integer from 2 to 10.

In another aspect, the compound is formula (VIII), wherein R⁹ is C1-C22 alkyl group or C2-C22 alkene group; R¹⁰ is C2-C200 alkyl group, C2-C200 heteroalkyl group, C2-C200 alkene group, or C2-C200 heteroalkene group; and f is an integer from 3 to 10.

In a further aspect, the compound is formula (VIII), wherein R⁹ is C1-C10 alkyl group; R¹⁰ is C2-C60 alkyl group, C2-C60 heteroalkyl group, C2-C60 alkene group, or C2-C60 heteroalkene group; and f is an integer from 3 to 10.

In an aspect, the compound is formula (VIII), wherein f is an integer from 3 to 6. In an aspect, the compound is formula (VIII), wherein f is 3, 4, 5, or 6.

In an aspect, the polycarboxylic acid ester is a product of a compound of formula (I), and/or a compound of formula (III) with a compound of formula (V).

In some aspects, the polycarboxylic acid ester may be selected from the group consisting of triethyl citrate, triethyl isocitrate, aconitic acid triethyl ester, propane-1,2,3-tricarboxylic acid triethyl ester, trimesic acid triethyl ester, carballylic acid triethyl ester, C54 trimer acid triethyl ester, mellitic acid hexaethyl ester, and combinations thereof. In a further aspect, the polycarboxylic acid ester may be selected from the group consisting of triethyl citrate, C54 trimer acid triethyl ester, and combinations thereof.

In an aspect, the at least one polycarboxylic acid ester may be a dicarboxylic acid ester. In some aspects, the dicarboxylic acid ester may be a compound of formula (IX)

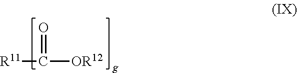

wherein
R¹¹ is C1-C22 alkyl group, C2-C22 alkene group, or C3-C22 cyclic group;
R¹² is C2-C200 alkyl group, C2-C200 heteroalkyl group, C2-C200 alkylene group, C2-C200 heteroalkene group, C2-C200 alkyne group, C2-C200 heteroalkyne group, C3-C200 cyclic group, or C2-C200 heterocyclic group; and
g is 2.

In an aspect, the compound is formula (IX), wherein R¹¹ is C1-C22 alkyl group or C2-C22 alkylene group; R¹² is C2-C200 alkyl group, C2-C200 heteroalkyl group, C2-C200 alkene group, or C2-C200 heteroalkene group; and g is 2. In a further aspect, the compound is formula (IX), wherein R¹¹ is C1-C10 alkyl group; R¹² is C2-C60 alkyl group, C2-C60 heteroalkyl group, C2-C60 alkene group, or C2-C60 heteroalkene group; and g is 2.

In some aspects, the dicarboxylic acid may be selected from the group consisting of diethyl malonate, diethyl succinate, diethyl fumarate, diethyl adipate, diethyl pimelate, diethyl suberate, diethyl azelate, diethyl sebacate, diethyl undecanedioate, diethyl dodecanedioate, diethyl tridecanedioate, diethyl hexadecanedioiate, C21 dimer acid diethyl ester, C36 dimer acid diethyl ester, hydrogenated C36 dimer acid diethyl ester, diethyl aspartate, diethyl glutamate, diethyl tartrate, diethyl malate, and combinations thereof. In a further aspect, the dicarboxylic acid ester may be selected from the group consisting of diethyl malonate, diethyl succinate, diethyl adipate, diethyl pimelate, diethyl azelate, diethyl sebacate, diethyl undecanedioate, C21 dimer acid diethyl ester, C36 dimer acid diethyl ester, hydrogenated C36 dimer acid diethyl ester, and combinations thereof.

In an aspect, the poly-carboxylic acid ester is biobased or naturally derived.

### 3. Polyol (ii)

In an aspect, the at least one polyol is a compound of formula (X)

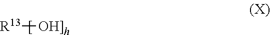

wherein
R¹³ is C2-C200 alkyl group, C2-C200 heteroalkyl group, C2-C200 alkene group, C2-C200 heteroalkene group, C2-C200 alkyne group, C2-C200 heteroalkyne group, C3-C200 cyclic group, or C2-C200 heterocyclic group; and
h is an integer from 2 to 10.

In an aspect, the compound is formula (X), wherein R¹³ is C2-C200 alkyl group, C2-C200 heteroalkyl group, C2-C200 alkene group, or C2-C200 heteroalkene group; and h is an integer from 2 to 10. In a further aspect, the compound is formula (X), wherein R¹³ is C2-C60 alkyl group, C2-C60 heteroalkyl group, C2-C60 alkene group, or C2-C60 heteroalkene group; and h is an integer from 2 to 10.

In an aspect, the compound is formula (X), wherein h is an integer from 2 to 6. In an aspect, the compound is formula (X), wherein h is 2, 3, 4, 5, or 6.

In an aspect, the polyol is a product of a compound of formula (I), and/or a compound of formula (III) with a compound of formula (V).

In an aspect, the C36 dimer diol is the diol produced from a C36 dimer acid.

In some aspects, the polyol may be selected from the group consisting of glycerol, diglycerol, polyglycerol, sorbitan, castor oil, hydrogenated castor oil, sugar alcohol, monosaccharide, disaccharides, oligosaccharide, polysaccharides, tannin, gallic acid, gluconic acid, lactobionic acid, gluconolactone, and combinations thereof. In a further aspect, the polyol may be selected from the group consisting of glycerol, diglycerol, polyglycerol, castor oil, hydrogenated castor oil, sorbitol, gallic acid, and combinations thereof. In another aspect, the alcohol may be selected from the group consisting of glycerol, diglycerol, polyglycerol, castor oil, hydrogenated castor oil, sorbitol, and combinations thereof.

In some aspects, the at least one polyol is a diol. In some aspects, the diol is a compound of formula (XI)

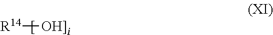

wherein
R¹⁴ is C2-C200 alkyl group, C2-C200 heteroalkyl group, C2-C200 alkene group, C2-C200 heteroalkene group, C2-C200 alkyne group, C2-C200 heteroalkyne group, C3-C200 cyclic group, or C2-C200 heterocyclic group; and
i is 2.

In an aspect, the compound is formula (XI), wherein R¹⁴ is C2-C200 alkyl group, C2-C200 heteroalkyl group, C2-C200 alkene group, or C2-C200 heteroalkene group; and i is 2. In a further aspect, the compound is formula (XI), wherein R¹⁴ is C2-C60 alkyl group, C2-C60 heteroalkyl group, C2-C60 alkene group, or C2-C60 heteroalkene group; and i is 2.

In some aspects, the diol may be selected from the group consisting of ethyleneglycol, 1,2-propanediol, 1,3-propanediol, 1,3-butanediol, 1,4-butanediol, 1,2-pentanediol, 1,3-pentanediol, 1,4-pentanediol, 1,5-pentanediol, 1,2-hexanediol, 1,5-hexanediol, 1,6-hexanediol, C36 dimer diol, and combinations thereof. In a further aspect, the diol may be selected from the group consisting of 1,3-propanediol, 1,3-butanediol, 1,4-butanediol, 1,5-pentanediol, 1,6-hexanediol, C36 dimer diol, hydrogenated C36 dimer diol, and combinations thereof. In one aspect, the diol may be selected from the group consisting of 1,3-propanediol, 1,3-butanediol, 1,4-butanediol, 1,5-pentanediol, 1,6-hexanediol, C36 dimer diol, hydrogenated C36 dimer diol, and combinations thereof.

In an aspect, the polyol is biobased or naturally derived.

### 4. Mono-carboxylic Acids

In an aspect, the at least one mono-carboxylic acid is a compound of formula (XII)

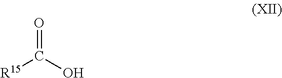

wherein

R¹⁵ is C2-C52 alkyl group, C2-C52 heteroalkyl group, C2-C52 alkene group, C2-C52 heteroalkene group, C3-C52 cyclic group, or C2-C52 heterocyclic group.

In an aspect, the mono-carboxylic acid is a compound of formula (XII), wherein R¹⁵ is C5-C21 alkyl group, C5-C21 heteroalkyl group, C5-C21 alkene group, C5-C21 heteroalkene group, C5-C21 cyclic group, or C5-C21 heterocyclic group.

In an aspect, the mono-carboxylic acid is selected from the group consisting of caprylic acid, capric acid, lauric acid, myristic acid, palmitic acid, stearic acid, isostearic acid, behenic acid, oleic acid, linoleic acid, linolenic acid, and combinations thereof. In an aspect, the mono-carboxylic acid is oleic acid. In an aspect, the mono-carboxylic acid is isostearic acid.

In an aspect, R¹⁵ is C2-C52 alkyl group with at least one hydroxyl (—OH) group.

In an aspect, the mono-carboxylic acid is selected from the group consisting of hydroxylstearic acid, ricinoleic acid, isoricinoleic acid, lesquerolic acid, densipolic acid, auricolic acid, dimorphecolic acid, and combinations thereof. In an aspect, the mono-carboxylic acid is hydroxylstearic acid, ricinoleic acid, and combinations thereof.

In an aspect, the mono-carboxylic acid is biobased or naturally derived.

### 5. Mono-Alcohols

In an aspect, the at least one mono-alcohol is a compound of formula (XIII)

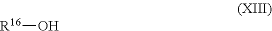

wherein
R¹⁶ is C2-C52 alkyl group, C2-C52 heteroalkyl group, C2-C52 alkene group, C2-C52 heteroalkene group, C3-C52 cyclic group, or C2-C52 heterocyclic group.

In an aspect, the mono-alcohol is a compound of formula (XIII), wherein R¹⁶ is C5-C21 alkyl group, C5-C21 heteroalkyl group, C5-C21 alkene group, C5-C21 heteroalkene group, C5-C21 cyclic group, or C5-C21 heterocyclic group.

In an aspect, the mono-alcohol is selected from the group consisting of octanol, decanol, lauryl alcohol, myristyl alcohol, cetyl alcohol, stearyl alcohol, isostearyl alcohol, behenyl alcohol, oleyl alcohol, linoleyl alcohol, linolenyl alcohol, and combinations thereof. In an aspect, the mono-alcohol is oleyl alcohol. In an aspect, the mono-alcohol is isostearyl alcohol.

In an aspect, the mono-alcohol is biobased or naturally derived.

B. Ratio of Components for the preparation of crosslinked polyester component (B)

In an aspect, the elastomer comprises a defined molar ratio of carboxyl functional groups (—COOH) from mono-carboxylic acid to hydroxyl functional groups (—OH) from polyol, and from hydroxyl functional groups (—OH) from mono-alcohol to carboxyl functional groups (—COOH) from poly-carboxylic acid. It has been found that the molar ratio of carboxyl functional groups (—COOH) from mono-carboxylic acid to hydroxyl functional groups (—OH) from the polyol and the molar ratio of hydroxyl functional groups (—OH) from mono-alcohol to carboxyl functional groups (—COOH) from poly-carboxylic acid influences the performance of the polyester elastomer and the performance of the gel made from the polyester elastomer.

In an aspect, the molar ratio of carboxyl functional groups (—COOH) from monocarboxylic acid to hydroxyl functional groups (—OH) from polyol is from about 1:2 to about 1:16. In an aspect, the molar ratio of carboxyl functional groups (—COOH) from mono-carboxylic acid to hydroxyl functional groups (—OH) from polyol is from about 1:2 to about 1:14. In an aspect, the molar ratio of carboxyl functional groups (—COOH) from mono-carboxylic acid to hydroxyl functional groups (—OH) from polyol is from about 1:2 to about 1:10. In an aspect, the molar ratio of carboxyl functional groups (—COOH) from mono-carboxylic acid to hydroxyl functional groups (—OH) from polyol is from about 1:2 to about 1:8. In an aspect, the molar ratio of carboxyl functional groups (—COOH) from mono-carboxylic acid to hydroxyl functional groups (—OH) from polyol is from about 1:2 to about 1:5. In an aspect, the molar ratio of carboxyl functional groups (—COOH) from mono-carboxylic acid to hydroxyl functional groups (—OH) from polyol is about 1:16, about 1:15, about 1:14, about 1:13, about 1:12, about 1:11, about 1:10, about 1:9, about 1:8, about 1:7, about 1:6, about 1:5, about 1:4, about 1:3, or about 1:2.

In an aspect, the molar ratio of hydroxyl functional groups (—OH) from mono-alcohol to carboxyl functional groups (—COOH) from poly-carboxylic acid is from about 1:2 to about 1:16. In an aspect, the molar ratio of hydroxyl functional groups (—OH) from mono-alcohol to carboxyl functional groups (—COOH) from poly-carboxylic acid is from about 1:2 to about 1:14. In an aspect, the molar ratio of hydroxyl functional groups (—OH) from mono-alcohol to carboxyl functional groups (—COOH) from poly-carboxylic acid is from about 1:2 to about 1:10. In an aspect, the molar ratio of hydroxyl functional groups (—OH) from mono-alcohol to carboxyl functional groups (—COOH) from poly-carboxylic acid is from about 1:2 to about 1:8. In an aspect, the molar ratio of hydroxyl functional groups (—OH) from mono-alcohol to carboxyl functional groups (—COOH) from poly-carboxylic acid is from about 1:2 to about 1:5. In an aspect, the molar ratio of hydroxyl functional groups (—OH) from mono-alcohol to carboxyl functional groups (—COOH) from poly-carboxylic acid is about 1:16, about 1:15, about 1:14, about 1:13, about 1:12, about 1:11, about 1:10, about 1:9, about 1:8, about 1:7, about 1:6, about 1:5, about 1:4, about 1:3, or about 1:2.

In an aspect, the molar ratio of carboxyl functional groups (—COOH) from poly-carboxylic acid to hydroxyl functional groups (—OH) from polyol is from about 1.5:1 to about 1:4. In an aspect, the ratio of carboxyl functional groups (—COOH) from poly-carboxylic acid to hydroxyl functional groups (—OH) from polyol is from about 1.5:1 to about 1:2. In an aspect, the ratio of carboxyl functional groups (—COOH) from poly-carboxylic acid to hydroxyl functional groups (—OH) from polyol is from about 1.5:1 to about 1:1.5. In an aspect, the ratio of carboxyl functional groups (—COOH) from poly-carboxylic acid to hydroxyl functional groups (—OH) from polyol is from about 1.5:1 to about 1:1.25. In an aspect, the ratio of carboxyl functional groups (—COOH) from poly-carboxylic acid to hydroxyl functional groups (—OH) from polyol is about 1.5:4, about 1.5:3, about 1.5:2, about 1:1, about 1:2, about 1:3, or about 1:4.

In an aspect, the conversion of carboxylic acid functional groups (—COOH) to ester functional groups (—CO(O)—) is no less than 80% by mole. The percent conversion is calculated by titration of carboxylic acid functional groups (—COOH) with 0.1 N KOH in isopropanol.

### C. Methods of Preparing the Polyester Elastomer (B)

### 1 Esterification Reaction

In one aspect, the present disclosure is directed to a method of preparing an elastomer (B) comprising reacting:
(i) at least one poly-carboxylic acid, at least one poly-carboxylic acid ester, or a combination thereof; and
(ii) at least one polyol; and/or
(iii) at least one mono-carboxylic acid; and/or
(iv) at least one mono-alcohol.

In an aspect, the elastomer prepared is a polyester elastomer (B). In an aspect, the elastomer prepared is a crosslinked polyester elastomer.

In an aspect, the preparation of the elastomer (B) is under nitrogen protection, is under vacuum, or is a combination thereof.

In an aspect, the elastomer is prepared by reacting:
(i) at least one poly-carboxylic acid, at least one poly-carboxylic acid ester, or a combination thereof; and
(ii) at least one polyol; and/or
(iii) at least one mono-carboxylic acid; and/or
(iv) at least one mono-alcohol.

In an aspect, wherein the reaction comprised an activated di-carboxylic acid or tricarboxylic acid, the preparation of the elastomer further comprises addition of water to quench the activating agent from the reaction.

In an aspect, esterification is conducted in solvent or emollient. In another aspect, esterification is conducted in more than one solvent or emollient.

In an aspect, esterification is carried out in the absence of a solvent or emollient.

In an aspect the crosslinked polyester component (B) is obtained by a process comprising the steps of:
(a) reacting compounds (i), (ii) and optionally (iii) as defined in the claims,
   optionally in the presence of one or more solvents, and
   optionally in the presence of one or more UV filter compounds (A),
to obtain a cross-linked polyester or a mixture thereof with the optional components,
b) optionally subjecting the crosslinked polyester or a mixture thereof with the optional components obtained in step (a) to a mechanical comminution process to obtain a cross-linked polyester component (B), or
c) optionally adding one or more solvents to the crosslinked polyester or a mixture thereof with the optional components obtained in step (a), allowing the resulting mixture to swell in said solvent, subjecting said swollen mixture to a mechanical comminution process and/or homogenization process in a reactor with planetary mixers to obtain a cross-linked polyester component (B) as a gel. In an aspect step (b) is carried out by mechanical stirring. In an aspect the mechanical comminution process step (c) is carried out with a three-roll mill.

So the several options to making the compositions of the invention include for example:
1. Mixing (A)+(B), where component (B) is made in the presence of a UV filter (A).
2 Mixing (A)+(B), where component (B) is made in the absence of a solvent.
3. Mixing (A)+(B), where component (B) is made in the presence of a solvent, and in the absence of UV filter (A).
4. Preparing (B) in the presence of a UV filter (A) (no solvent).
5. Preparing (B) in the presence of a UV filter (A) and a solvent.
6. Preparing (B) in the presence of a UV filter (A) and another ingredient(s) commonly used in cosmetics (D) (no solvent).
7. Preparing (B) in the presence of a UV filter (A), a solvent and another ingredient(s) commonly used in cosmetics (D).

### 2. Ratio of Components

In an aspect, the preparation of the elastomer comprises a defined ratio of poly-carboxylic acid, poly-carboxylic acid ester or a combination thereof (i) to polyol (ii), a defined ratio of monocarboxylic acid (iii) (a) to polyol (ii), and a defined ratio of mono-alcohol (iii) (b) to poly-carboxylic acid, poly-carboxylic acid ester, or a combination thereof (i). It has been found that the ratio of (i)/(iii) (a), (ii)/(iii) (a), and (iii) (b)/(i) influences the performance of the polyester elastomer and the performance of the gel made from the polyester elastomer.

In an aspect, the molar ratio of carboxyl functional groups (—COOH) from monocarboxylic acid (iii) (a) to hydroxyl functional groups (—OH) from polyol (ii) is from about 1:2 to about 1:16. In an aspect, the molar ratio of carboxyl functional groups (—COOH) from mono-carboxylic acid (iii) (a) to hydroxyl functional groups (—OH) from polyol (ii) is from about 1:2 to about 1:14. In an aspect, the molar ratio of carboxyl functional groups (—COOH) from mono-carboxylic acid (iii) (a) to hydroxyl functional groups (—OH) from polyol (ii) is from about 1:2 to about 1:10. In an aspect, the molar ratio of carboxyl functional groups (—COOH) from mono-carboxylic acid (iii) (a) to hydroxyl functional groups (—OH) from polyol (ii) is from about 1:2 to about 1:8. In an aspect, the molar ratio of carboxyl functional groups (—COOH) from mono-carboxylic acid (iii) (a) to hydroxyl functional groups (—OH) from polyol (ii) is from about 1:2 to about 1:5. In an aspect, the molar ratio of carboxyl functional groups (—COOH) from mono-carboxylic acid (iii) (a) to hydroxyl functional groups (—OH) from polyol (ii) is about 1:16, about 1:15, about 1:14, about 1:13, about 1:12, about 1:11, about 1:10, about 1:9, about 1:8, about 1:7, about 1:6, about 1:5, about 1:4, about 1:3, or about 1:2.

In an aspect, the molar ratio of carboxyl functional groups (—COOH) from poly-carboxylic acid, poly-carboxylic acid ester or a combination thereof (i) to hydroxyl functional groups (—OH) from polyol (ii) is from about 1.5:1 to about 1:4. In an aspect, the ratio of carboxyl functional groups (—COOH) from poly-carboxylic acid, poly-carboxylic acid ester or a combination thereof (i) to hydroxyl functional groups (—OH) from polyol (ii) is from about 1.5:1 to about 1:2. In an aspect, the ratio of carboxyl functional groups (—COOH) poly-carboxylic acid, poly-carboxylic acid ester or a combination thereof (i) to hydroxyl functional groups (—OH) from polyol (ii) is from about 1.5:1 to about 1:1.5. In an aspect, the ratio of carboxyl functional groups (—COOH) poly-carboxylic acid, poly-carboxylic acid ester or a combination thereof (i) to hydroxyl functional groups (—OH) from polyol (ii) is from about 1.5:1 to about 1:1.25. In an aspect, the ratio of carboxyl functional groups (—COOH) poly-carboxylic acid, poly-carboxylic acid ester or a combination thereof (i) to hydroxyl functional groups (—OH) from polyol (ii) is about 1.5:4, about 1.5:3, about 1.5:2, about 1:1, about 1:2, about 1:3, or about 1:4.

In an aspect, the molar ratio of hydroxyl functional groups (—OH) from mono-alcohol (iii) (b) to carboxyl functional groups (—COOH) from poly-carboxylic acid (i) is from about 1:2 to about 1:16. In an aspect, the molar ratio of hydroxyl functional groups (—OH) from mono-alcohol (iii) (b) to carboxyl functional groups (—COOH) from poly-carboxylic acid (i) is from about 1:2 to about 1:14. In an aspect, the molar ratio of hydroxyl functional groups (—OH) from mono-alcohol (iii) (b) to carboxyl functional groups (—COOH) from poly-carboxylic acid (i) is from about 1:2 to about 1:10. In an aspect, the molar ratio of hydroxyl functional groups (—OH) from mono-alcohol (iii) (b) to carboxyl functional groups (—COOH) from poly-carboxylic acid (i) is from about 1:2 to about 1:8. In an aspect, the molar ratio of hydroxyl functional groups (—OH) from mono-alcohol (iii) (b) to carboxyl functional groups (—COOH) from poly-carboxylic acid (i) is from about 1:2 to about 1:5. In an aspect, the molar ratio of hydroxyl functional groups (—OH) from mono-alcohol (iii) (b) carboxyl functional groups (—COOH) from poly-carboxylic acid (A) is about 1:16, about 1:15, about 1:14, about 1:13, about 1:12, about 1:11, about 1:10, about 1:9, about 1:8, about 1:7, about 1:6, about 1:5, about 1:4, about 1:3, or about 1:2.

In an aspect, the conversion of carboxyl functional groups (—COOH) to ester functional groups (—CO(O)—) is no less than 80% by mole. The percent conversion is calculated by titration of carboxylic acid functional groups (—COOH) with 0.1N KOH in isopropanol.

In an aspect, esterification is carried out in the absence of a solvent or emollient.

In an aspect, esterification is conducted in solvent or emollient. In another aspect, esterification is conducted in more than one solvent or emollient.

In an aspect, the percentage of solvent or emollient in total raw materials of the esterification reaction is in the range of 0% to 80% by weight. In an aspect, the percentage of solvent or emollient in total raw materials of the esterification reaction is in the range of 0% to 60% by weight. In an aspect, the percentage of solvent or emollient in total raw materials of the esterification reaction is in the range of 0% to 50% by weight. In an aspect, the percentage of solvent or emollient in total raw materials of the esterification reaction is in the range of 0% to 40% by weight. In an aspect, the percentage of solvent or emollient in total raw materials of the esterification reaction is in the range of 0% to 30% by weight. In an aspect, the percentage of solvent or emollient in total raw materials of the esterification is in the range of 0% to 20% by weight. In an aspect, the percentage of solvent or emollient in total raw materials of the esterification reaction is in the range of 0% to 10% by weight. In an aspect, the percentage of solvent or emollient in total raw materials of the esterification reaction is in the range of 10% to 20% by weight. In an aspect, the percentage of solvent or emollient in total raw materials of the esterification reaction is in the range of 20% to 30% by weight.

### 3 Activating Agent

In an aspect, the preparation of the elastomer further comprises an activating agent. In an aspect, the preparation of the elastomer does not comprise an activating agent.

In an aspect, the activating agent is selected from the group consisting of dimethyl dicarbonate, diethyl dicarbonate, dipropyl dicarbonate, di-tertiary-butyl dicarbonate, and combinations thereof.

### 4 Catalyst

In an aspect, the preparation of the elastomer further comprises a catalyst. In an aspect, the preparation of the elastomer does not comprise a catalyst. However, it has been found that when no catalyst is used the reaction times are protracted.

In an aspect, the catalyst is selected from the group consisting of methanesulfonic acid, p-toluenesulfonic acid, benzene sulfonic acid, sulfuric acid, amidosulfonic acid, sulfamic acid, sodium bisulfate, phosphoric acid, hydrochloric acid, hydrobromic acid, nitric acid, sodium hydroxide, potassium hydroxide, sodium methoxide, sodium ethoxide, potassium methoxide, potassium ethoxide, bismuth (III) neodecanoate, bismuth (III) citrate, bismuth (III) chloride, bismuth (III) acetate, bismuth (III) phosphate, tin chloride, tin-pyrone, dibutyltin dilaurate, di-n butyl-oxo-stannane, butyl stannoic acid, zinc chloride, zinc bromide, zinc carboxylic salt, zinc oxide, zinc hydroxy nitrate salt, zinc hydroxy acetate, triethylamine, tripropylamine, cocamidopropyl dimethylamine, stearamidopropyl dimethylamine, isostearamidopropyl dimethylamine, and combinations thereof. In an aspect, the catalyst is p-toluenesulfonic acid, methanesulfonic acid, phosphoric acid, bismuth neodecanoate, or a combination thereof. In an aspect, the catalyst is methanesulfonic acid.

In an aspect, the catalyst is a salt. In an aspect, the catalyst is a salt selected from the group consisting of Yb(OTf)₃, Sc(OTf)₃, Hf(OTf)₄, Bi(OTf)₃, Al(OTf)₃, Zn(OTf)₂, Mg(ClO4)₂, Cu(OTf)₂, Ti(OCH(CH₃)₂)₄, and combinations thereof.

### 5. Emollient or Solvent

In an aspect the cosmetically acceptable carrier (C) is preferably selected from the emollients or solvents as described herein below. The cosmetically acceptable carrier (C) can be present in the preparation of the crosslinked polyester component (B) and/or can be added after the preparation of the crosslinked polyester component (B).

In an aspect, the preparation of the polyester elastomer (B) can occur in the presence of a solvent. The solvent can also act as an emollient, preferably a cosmetic emollient. When the solvent acts also as an emollient it also provides a softening, protecting, moisturizing, and/or lubricating effect to the skin. In an aspect, the solvent or emollient is preferably a biobased or naturally derived. In an aspect, the solvent or emollient is a triglyceride solvent, a mono-ester solvent, a di-ester solvent, a citrate ester solvent, an ether solvent, a carbonate solvent, a hydrocarbon solvent, a silicone solvent, or a combination thereof.

In an aspect, the solvent is a triglyceride solvent of formula (XIV)

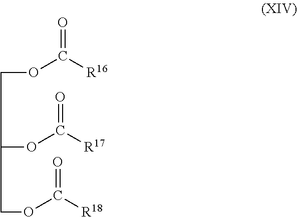

wherein
each R¹⁶, R¹⁷, and R¹⁸ are independently C1-C35 alkyl group, C1-C35 heteroalkyl group, C2-C35 alkene group, or C2-C35 heteroalkene group.

In an aspect, the solvent is of formula (XIV), wherein R¹⁶, R¹⁷, and R¹⁸ are independently C2-C17 alkyl group or C2-C17 alkylene group.

In an aspect, the solvent is a triglyceride solvent selected from the group consisting of caprylic/capric triglyceride, triheptanoin, corn oil, soybean oil, olive oil, rape seed oil, cotton seed oil, coconut oil, almond oil, argon oil, rosehip oil, black seed oil, grape seed oil, avocado oil, apricot kernel oil, geranium oil, lavender oil, rosehip oil, macadamia oil, eucalyptus oil, sardine oil, herring oil, safflower oil, linseed oil, sunflower oil, olive oil, canola oil, sesame oil, cottonseed oil, palm oil, rapeseed oil, tung oil, fish oil, peanut oil, cuphea oil, milkweed oil, salicornia oil, whale oil, castor oil, and combinations thereof. In an aspect, the triglyceride solvent is selected from the group consisting of caprylic/capric triglyceride, triheptanoin, and combinations thereof.

In an aspect, the solvent is a mono-ester solvent of formula (XV)

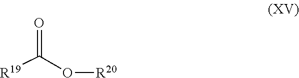

wherein
R¹⁹ is C1-C35 alkyl group, C1-C35 heteroalkyl group, C2-C35 alkene group, or C2-C35 heteroalkene group; and

R²⁰ is C1-C35 alkyl group, C1-C35 heteroalkyl group, C2-C35 alkene group, or C2-C35 heteroalkene group.

In an aspect, the solvent is a mono-ester solvent of formula (XV), wherein R¹⁹ is C5-C17 alkyl group or C5-C17 alkene group and R²⁰ is C2-C17 alkyl group or C2-C17 alkene group.

In an aspect, the solvent is a mono-ester solvent selected from the group consisting of coco-caprylate, coco-caprate, jojoba oil, jojoba esters, isopropyl jojobate, ethyl macadamiate, isoamyl laurate, heptyl undecylenate, methylheptyl isostearate, isostearyl isostearate, glyceryl ricinoleate, isostearyl palmitate, myristyl myristate, octyldodecyl myristate, octyldodecyl hydroxystearate, butyl myristate, ethylhexyl cocoate, ethylhexyl palmitate, ethylhexyl stearate, butyl stearate, decyl oleate, isocetyl behenate, isocetyl myristate, isocetyl palmitate, isocetyl stearate, isodecyl oleate, isopropyl isostearate, isopropyl myristate, isopropyl palmitate, oleyl oleate, propylene glycol laurate, octyldodecyl erucate, C12-C13 alkyl lactate, C12-C15 alkyl lactate, isostearyl lactate, glycereth-5-lactate, lauryl lactate, myristyl lactate, oleyl lactate, laureth-2-benzoate, C12-C15 alkyl benzoate, C12-C15 pareth-3-benzoate, dipropylene glycol benzoate, isodecyl salicylate, C12-C15 alkyl salicylate, tridecyl salicylate, ethylhexyl isononanoate, cetyl ethylhexanoate, isononyl isononanoate, isodecyl ethylhexanoate, isodecyl isononanoate, tridecyl ethylhexanoate, isotridecyl isononanoate, isostearyl isononanoate, cetearyl isononanoate, laureth-2-ethylhexanoate, cetearyl ethylhexanoate, isodecyl neopentanoate, isostearyl neopentanoate, nyristyl neopentanoate, isostearyl behenate, octyldodecyl neopentanoate, tridecyl neopentanoate, and combinations thereof. In an aspect, the mono-ester solvent is selected from the group consisting of cococaprylate/caprate, coco-caprylate, jojoba oil, isoamyl laurate, methylheptyl isostearate, C12-C13 alkyl lactate, C12-C15 alkyl lactate, lauryl lactate, ethylhexyl isononanoate, cetyl ethylhexanoate, isononyl isononanoate, isodecyl ethylhexanoate, isodecyl isononanoate, tridecyl ethylhexanoate, isotridecyl isononanoate, isostearyl isononanoate, cetearyl isononanoate, and combinations thereof. In an aspect, the mono-ester solvent is selected from the group consisting of coco-caprylate/caprate, coco-caprylate, isoamyl laurate, isononyl isononanoate, heptyl undecylenate, jojoba oil, jojoba esters, and combinations thereof.

In an aspect, the solvent is:
(a) a di-ester solvent of formula (XVI)

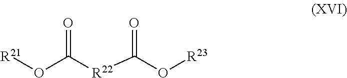

wherein
R²¹ is C₁-C35 alkyl group, C1-C35 heteroalkyl group, C2-C35 alkene group, or C2-C35 heteroalkene group; and
R²² and R²³ are independently C1-C35 alkyl group, C1-C35 heteroalkyl group, C2-C35 alkene group, or C2-C35 heteroalkene group; or
(b) a di-ester solvent of formula (XVII)

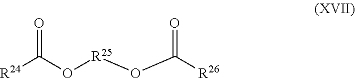

wherein
R²⁴ is C1-C35 alkyl group, C1-C35 heteroalkyl group, C2-C35 alkene group, or C2-C35 heteroalkene group; and
R²⁵ and R²⁶ are independently H, C1-C35 alkyl group, C1-C35 heteroalkyl group, C2-C35 alkene group, or C2-C35 heteroalkene group; or
(c) a di-ester solvent of formula (XVIII)

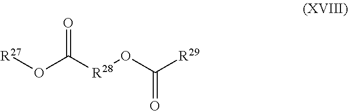

wherein
R²⁷ and R²⁸ are independently C1-C35 alkyl group, C1-C35 heteroalkyl group, C2-C35 alkene group, or C2-C35 heteroalkene group; and
R²⁹ is H, C1-C35 alkyl group, C1-C35 heteroalkyl group, C2-C35 alkene group, or C2-C35 heteroalkene group.

In an aspect, the solvent is a di-ester solvent of formula (XVI), formula (XVII), or formula (XVIII), wherein R²³, R²⁶, and R²⁹ is C2-C10 alkyl group or C2-C10 alkene group and R²¹, R²⁴, R²⁷, and R²², R²⁵, and R²⁸ are independently C1-C12 alkyl group or C2-C12 alkene group.

In an aspect, the di-ester solvent is selected from the group consisting of diethyl succinate, dibutyl succinate, diethyhexyl succinate, diisopropyl sebacate, dimethyl sebacate, diethyl sebacate, dibutyl sebacate, diisostearyl dimer, diisostearyl malate, isostearyl stearoyl stearate, isocetyl stearoyl stearate, octyldodecyl stearoyl stearate, diethylhexyl malate, diethylhexyl maleate, dipropylene glycol dibenzoate, dicapryl adipate, dicaprylyl maleate, diisopropyl dimer, diisopropyl adipate, diisobutyl adipate, diisopropyl sebacate, diisostearyl dimer, diethyhexyl succinate, diethylene glycol diethylhexanoate, neopentyl glycol dicaprate, propylene glycol dicaprylate/dicaprate, neopentyl glycol diisostearate, neopentyl glycol diethylhexanoate, neopentyl glycol diheptanoate, and combinations thereof. In an aspect, the di-ester solvent is selected from the group consisting of dicapryl adipate, dicaprylyl maleate, diisopropyl adipate, diisobutyl adipate, diethyl succinate, dibutyl succinate, diethyhexyl succinate, diisopropyl sebacate, dimethyl sebacate, diethyl sebacate, dibutyl sebacate, neopentyl glycol diethylhexanoate, neopentyl glycol diheptanoate, and combinations thereof.

In an aspect, the solvent is a citrate ester solvent of formula (XVIII)

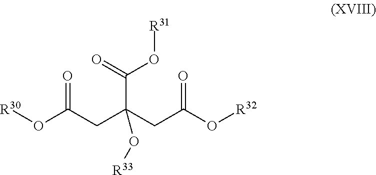

wherein
R³⁰, R³¹, R³², and R³³ are independently H, C1-C35 alkyl group, C1-C35 heteroalkyl group, C2-C35 alkene group, or C2-C35 heteroalkene group, wherein at least one of R³⁰, R³¹, R³², and R³³ is not H.

In an aspect, the solvent is a citrate ester solvent of formula (XVIII), wherein R³⁰, R³¹, and R³² are independently C1-C10 alkyl group or C2-C10 alkene group and R³³ is an acetyl group.

In an aspect, the solvent is a citrate ester solvent selected from the group consisting of tricaprylyl citrate, triisostearyl citrate, triisocetyl citrate, trioctyldodecyl citrate, triethyl citrate, tributyl citrate, acetyl triethyl citrate, acetyl tributyl citrate, trioctyldodecyl citrate, triisocetyl citrate, and combinations thereof.

In an aspect, the solvent is an ether solvent of formula (XIX)

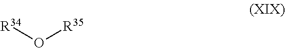

wherein
R³⁴ and R³⁵ are independently H, C2-C20 alkyl group, C2-C20 heteroalkyl group, C2-C20 alkene group, or C2-C20 heteroalkene group, wherein at least one of R³⁴ and R³⁵ is not H.

In an aspect, the solvent is an ether solvent of formula (XIX), wherein R³⁴ and R³⁵ are independently C2-C20 alkyl group.

In an aspect, the solvent is an ether solvent selected from the group consisting of dicaprylyl ether, didecyl ether, panthenyl ethyl ether, dicetyl ether, dimyristyl ether, distearyl ether, dilauryl ether, and combinations thereof. In an aspect, the ether solvent is selected from the group consisting of dicaprylyl ether, didecyl ether, and combinations thereof.

In an aspect, the solvent is a carbonate solvent of formula (XX)

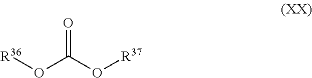

wherein
R³⁶ and R³⁷ are independently H, C2-C20 alkyl group, C2-C20 heteroalkyl group, C2-C20 alkene group, or C2-C20 heteroalkene group.

In an aspect, the solvent is a carbonate solvent of formula (XX), wherein R³⁶ and R³⁷ are independently C2-C20 alkyl group.

In an aspect, the solvent is a carbonate solvent selected from the group consisting of dicaprylyl carbonate, diethyl hexyl carbonate, and combinations thereof.

In an aspect, the solvent is a hydrocarbon with number of carbon atoms from C4 to C60. In an aspect, the solvent is a hydrocarbon with a number of carbon atoms from C10 to C50. In an aspect, the solvent is a hydrocarbon with a number of carbon atoms from C20 to C40.

In an aspect, the solvent is a hydrocarbon solvent selected from the group consisting of farnesene, hydrogenated farnesene, coconut alkanes, coconut/palm kernel alkanes, C9-C12 alkane, C10-C13 alkane, C12-C17 alkane, C13-C14 alkane, C13-C15 alkane, C14-C17 alkane, C14-C19 alkane, C14-C20 alkane, C14-C22 alkane, C15-C19 alkane, C21-C28 alkane, C17-C23 alkane, C9-C12 isoalkane, C9-C13 isoalkane, C9-C14 isoalkane, C9-C16 isoalkane, C10-C11 isoalkane, C10-C12 isoalkane, C10-C13 isoalkane, C11-C12 isoalkane, C11-C13 isoalkane, C11-C14 isoalkane, C12-C14 isoalkane, C12-C15 isoalkane, C12-C20 isoalkane, C13-C14 isoalkane, C13-C16 isoalkane, C14-C16 isoalkane, C15-C19 isoalkane, C10-C16 olefin, C12-C18 olefin, C18-C26 olefin, C20 olefin, C20-C24 olefin, C24-C30 olefin, C26-C28 olefin, C26-C54 olefin, C28-C36 olefin, C28-C52 olefin, C30-C38 olefin, C30-C45 olefin, C4-C12 olefin, C4-C6 olefin, C5-C6 olefin, hydrogenated poly(C6/C10/C14 olefin), hydrogenated poly(C6-C12 olefin), hydrogenated poly(C6-C14 olefin), hydrogenated poly(C6-C20 olefin), hydrogenated poly(C8/C12 olefin), poly(C20-C28 olefin), poly(C30-C45 olefin), poly(C4-C12 olefin), poly(C6-C14 olefin), hexadecene, C32 alkane, C32 isoalkane, C54 alkane, C54 isoalkane, diethylhexylcyclohexane, undecane, tridecane, tetradecane, pentadecane, hexadecane, octadecane, docosane, squalane, hydrogenated polyisobutene, polybutene, hydrogenated polydecene, hydrogenated didecene, mineral oil, liquidum, petrolatum, dodecane, isohexadecane, isododecane, isoeicosane, and combinations thereof. In an aspect, the hydrocarbon solvent is selected from the group consisting of squalane, farnesene, hydrogenated farnesene, coconut alkanes, C9-C12 alkane, C13-C15 alkane, C14-C19 alkane, C14-C20 alkane, C14-C22 alkane, C15-C19 alkane, C13-C16 isoalkane, dodecane, undecane, tridecane, tetradecane, pentadecane, hexadecane, hexadecene, octadecane, squalane, isododecane, isohexadecane, C32 alkane, C32 isoalkane, C54 alkane, C54 isoalkane, and combinations thereof. In an aspect, the hydrocarbon solvent is selected from the group consisting of squalane, hydrogenated farnesene, coconut alkanes, C9-C12 alkane, C13-C15 alkane, C13-C16 isoalkane, C14-C19 alkane, dodecane, tetradecane, isododecane, hexadecane, octadecane, hexadecene, C32 alkane, C32 isoalkane, C54 alkane, C54 isoalkane, and combinations thereof.

In an aspect, the hydrocarbon solvent is selected from the group consisting of squalane, C32 alkane, C32 isoalkane, C54 alkane, C54 isoalkane, and combinations thereof.

In an aspect, the solvent may be a chemical UV filter. The UV filter solvent may be selected from the group consisting of ethyl dimethyl PABA, ethylhexyl methoxycinnamate, ethylhexyl salicylate, homosalate, isoamyl p-Methoxycinnamate, menthyl anthranilate, octocrylene, polysilicone-15, terepthalyidene dicamphor sulfonic acid, triethanolamine salicylate, and combinations thereof.

In an aspect, the solvent is a silicone solvent selected from the group consisting of dimethicone, phenyl dimethicone, caprylyl methicone, ethyl trisiloxane, cyclotetrasiloxane, cyclopentasiloxane, cyclohexasiloxane, and combinations thereof.

In an aspect, a defined amount of solvent is used in the preparation of the polyester elastomer. In an aspect, the amount of solvent is from 0% to 70% of the total weight of poly-carboxylic acid (i), polyol (ii), mono-carboxylic acid (iii) (a) and mono-alcohol (iii) (b). In an aspect, the amount of solvent is from 0% to 50% of the total weight of poly-carboxylic acid (i), polyol (ii), mono-carboxylic acid (iii) (a) and mono-alcohol (iii) (b). In an aspect, the amount of solvent is from 0% to 40% of the total weight of poly-carboxylic acid (i), polyol (ii), mono-carboxylic acid (iii) (a) and mono-alcohol (iii) (b). In an aspect, the amount of solvent is from 0% to 30% of the total weight poly-carboxylic acid (i), polyol (ii), mono-carboxylic acid (iii) (a) and mono-alcohol (iii) (b). In an aspect, the range of solvent is from 0% to 20% of the total weight of poly-carboxylic acid (i), polyol (ii), mono-carboxylic acid (iii) (a) and mono-alcohol (iii) (b). In an aspect, the amount of solvent is from 10% to 50% of the total weight of poly-carboxylic acid (i), polyol (ii), mono-carboxylic acid (iii) (a) and mono-alcohol (iii) (b). In an aspect, the amount solvent is 50%, 40%, 30%, 20%, or 10% of the total weight of poly-carboxylic acid (i), polyol (ii), mono-carboxylic acid (iii) (a) and mono-alcohol (iii) (b).

In an aspect, the amount of solvent is from 0% to 30% of the total weight of poly-carboxylic acid (i) and mono-carboxylic acid (iii) (a). In an aspect, the amount of solvent is from 0% to 20% of the total weight of poly-carboxylic acid (i) and mono-carboxylic acid (iii) (a). In an aspect, the amount of solvent is from 0% to 10% of the total weight of poly-carboxylic acid (i) and mono-carboxylic acid (iii) (a). In an aspect, the amount of solvent is from 10% to 30% of the total weight of poly-carboxylic acid (i) and mono-carboxylic acid (iii) (a). In an aspect, the solvent is 30%, 20%, or 10% of the total weight of poly-carboxylic acid (i) and mono-carboxylic acid (iii) (a).

In an aspect, no solvent is used to prepare the elastomer (B).

In an aspect, a solvent is used to prepare the polyester elastomer (B), which is removed after preparing the polyester elastomer (B) to form a polyester elastomer (B) powder. In an aspect, to the polyester elastomer powder a solvent/emollient can be added again to form a polyester elastomer (B) gel, optionally applying a shear force as described below.

In an aspect, polyester elastomer (B) is made from C36 dimer acid, diglycerol, and isostearic acid with about 10% to about 40% by weight squalane based on the total weight of the polyester elastomer (B) and the squalane. That is, in an aspect the invention relates to a composition of a polyester elastomer made from C36 dimer acid, diglycerol, isostearic acid, and squalane, comprising about 10% to about 40% by weight squalane.

In an aspect, polyester elastomer (B) is made from hydrogenated C36 dimer acid, diglycerol, and oleic acid without any solvent or emollient.

### 6. Temperature

In an aspect, the method of preparing the elastomer comprises reacting at least one poly-carboxylic acid, poly-carboxylic acid ester, or combination thereof, at least one polyol, and/or at least one mono-carboxylic acid, and/or at least one mono-alcohol with mixing at a pre-determined temperature until an elastomer is formed. In an aspect, the method of preparing the elastomer comprises reacting at least one poly-carboxylic acid, poly-carboxylic acid ester, or combination thereof, at least one polyol, and/or at least one mono-carboxylic acid, and/or at least one mono-alcohol, optionally at least one solvent or emollient, and optionally a catalyst with mixing at a pre-determined temperature until an elastomer is formed. In an aspect, the temperature range is from 30° C. to 250° C.

In an aspect, the reaction occurs at a temperature from about 30° C. to about 250° C. In an aspect, the reaction occurs at a temperature from about 60° C. to about 250° C. In an aspect, the reaction occurs at a temperature from about 30° C. to about 125° C. or about 40° C. to about 100° C. In an aspect, the reaction occurs at a temperature of about 30° C., about 35° C., about 40° C., about 45° C., about 50° C., about 55° C., about 60° C., about 65° C., about 70° C., about 75° C., about 80° C., about 85° C., about 90° C., about 95° C., about 100° C., about 105° C., about 110° C., about 115° C., about 120° C., about 125° C., about 130° C., about 135° C., about 140° C., about 145° C., about 150° C., about 155° C., about 160° C., about 165° C., about 170° C., about 175° C., about 180° C., about 185° C., about 190° C., about 195° C., about 200° C., about 205° C., about 210° C., about 215° C., about 220° C., about 225° C., about 230° C., about 235° C., about 240° C., about 245° C., or about 250° C.

### 7. Time

In an aspect, the reaction time is from about 12 hours to about 150 hours. In an aspect, the reaction time is from about 6 hours to about 24 hours. In an aspect, the reaction time is from about 8 hours to about 27 hours. In an aspect, the reaction time is about 6 hours, about 6.5 hours, about 7 hours, about 7.5 hours, about 8 hours, about 8.5 hours, about 9 hours, about 9.5 hours, about 10 hours, about 10.5 hours, about 11 hours, about 11.5 hours, about 12 hours, about 12.5 hours, about 13 hours, about 13.5 hours, about 14 hours, about 14.5 hours, about 15 hours, about 15.5 hours, about 16 hours, about 16.5 hours, about 17 hours, about 17.5 hours, about 18 hours, about 18.5 hours, about 19 hours, about 19.5 hours, about 20 hours, about 20.5 hours, about 21 hours, about 21.5 hours, about 22 hours, about 22.5 hours, about 23 hours, about 23.5 hours, about 24 hours, about 24.5 hours, about 25 hours, about 25.5 hours, about 26 hours, about 26.5 hours, about 27 hours, about 28 hours, about 29 hours, about 30 hours, about 31 hours, about 32 hours, about 33 hours, about 34 hours, about 35 hours, about 36 hours, about 37 hours, about 38 hours, about 39 hours, about 40 hours, about 41 hours, about 42 hours, about 43 hours, about 44 hours, about 45 hours, about 46 hours, about 47 hours, about 48 hours, about 49 hours, about 50 hours, about 55 hours, about 60 hours, about 65 hours, about 70 hours, about 75 hours, about 80 hours, about 85 hours, about 90 hours, about 95 hours, about 100 hours, about 105 hours, about 110 hours, about 115 hours, about 120 hours, about 125 hours, about 130 hours, about 135 hours, about 140 hours, about 145 hours, or about 150 hours.

The reaction time can be adjusted by determination of the gel fraction achieved, and preferably the reaction time is such that the gel fraction of the polyester elastomer is greater than 60%. A method to measure the gel fraction in the polyester elastomer is described below.

### 8. By-Product Removal

In an aspect, the method further comprises removing water and alcohol by-product from the reaction. In a further aspect, the water and alcohol by-products are removed from the reaction by mixing and heating the reaction. In an aspect, the reaction is heated to above about 120° C. to remove the water and alcohol by-products. In an aspect, the water and alcohol by-products are removed from the reaction by nitrogen flow, by vacuum, or a combination thereof. In an aspect, water is removed by nitrogen stripping and vacuum, which have an impact on the reaction time.

As mentioned above in an aspect, the polyester elastomer (B) is provided as a (at room temperature (23° C.) flowable or fluid) gel or a (dry) powder.

In an aspect, the polyester elastomer (B) is processed into a gel as described herein.

In an aspect, polyester elastomer (B) used is only comprised of crosslinked polyester without solvent or emollient. In an aspect, polyester elastomer (B) used is comprised of polyester without solvent or emollient. In an aspect, polyester elastomer (B) used is comprised of crosslinked polyester with solvent or emollient. In an aspect, polyester elastomer (B) used is comprised of polyester with solvent or emollient.

Solvents or emollients that can be used to prepare the polyester elastomer composition (B) are as described herein above and can be selected from the solvents or emollients as defined herein. In another aspect, the polyester elastomer in polyester elastomer composition (B) used is in the range of 5 wt % to 100 wt %. In another aspect, the polyester elastomer in polyester elastomer composition (B) used is in the range of 5 wt % to 70 wt %. In another aspect, the polyester elastomer in polyester elastomer composition (B) used is in the range of 10 wt % to 60 wt %. In another aspect, the polyester elastomer in polyester elastomer composition (B) used is in the range of 20 wt % to 50 wt %.

In another aspect, the crosslinked polyester in polyester elastomer composition (B) used is in the range of 5 wt % to 50 wt %. In another aspect, the crosslinked polyester in polyester elastomer composition (B) used is in the range of 5 wt % to 30 wt %. In another aspect, the crosslinked polyester in polyester elastomer composition (B) used is in the range of 10 wt % to 30 wt %. Solvents or emollients that can be used to prepare the polyester elastomer composition are described herein and can be selected from the solvents or emollients as defined herein.

In an aspect, the polyester elastomer composition (B) used comprises at least one solvent or emollient added to the polyester elastomer during the shearing force process to form a gel. Solvents or emollients that can be used to prepare the polyester elastomer composition are described herein and can be selected from the solvents as defined herein. In an aspect, the solvent or emollient is from about 20% to about 95% weight by weight of the composition. In an aspect, the solvent or emollient is from about 20% to about 50% weight by weight of the composition. In an aspect, the solvent or emollient is from about 50% to about 90% weight by weight of the composition. In an aspect, the solvent or emollient is from about 70% to about 90% weight by weight of the composition. In an aspect, the polyester elastomer composition (B) comprises from about 50% to about 90% weight by weight of solvent or emollient, from about 50% to about 80% weight by weight of solvent or emollient, from about 50% to about 70% weight by weight of solvent or emollient, or from about 50% to about 60% weight by weight of solvent or emollient. In some embodiments, the polyester elastomer composition (B) comprises about 20%, about 30%, about 40%, about 50%, about 60%, about 70%, about 80%, or about 90% weight by weight of solvent or emollient.

### Process to Prepare the Polyester Elastomer Component (B) as a Gel Composition

In an aspect, the polyester elastomer obtained by the esterification process is crumbled or processed to form a polyester elastomer powder. In another aspect, the polyester elastomer is processed by a three-roll mill to form a polyester elastomer powder.

The polyester elastomer composition (B) can be also obtained as a powder if it still comprises a solvent or emollient added to the reaction mixture.

In an aspect, polyester elastomer composition (B) is prepared by combining the polyester elastomer with one or more solvents or emollients to form a polyester elastomer gel.

In an aspect, polyester elastomer and solvent/emollient mixture are processed with a homogenizer to produce a gel optionally applying a shear force, e.g. by a high-shear disperser mixer.

In an aspect to prepare the polyester elastomer composition (B) as gel, a polyester elastomer is swelled in a solvent or emollient before being processed to make a gel at a temperature under 23° C. In an aspect, the time of polyester elastomer swelling in a solvent or emollient is from 1 hour to 1 week. In an aspect, the elastomer is subject to swelling in a solvent or emollient from 10 minutes to 1 week, from 10 minutes to 4 days, from 10 minutes to 3 days, from 10 minutes to 2 days, from 10 minutes to 1 day, from 10 minutes to 12 hours, from 10 minutes to 6 hours, from 10 minutes to 3 hours, from 10 minutes to 2 hours, from 10 minutes to 1 hour, or from 10 minutes to 30 minutes.

Once the initially produced polyester elastomer is prepared, it can be mixed with an additional quantity of at least one solvent or emollient that can be different from the solvent emollient used to prepare the initially produced elastomer. In some aspects, the at least one solvent or emollient used to prepare the elastomer is the same as the at least one solvent or emollient used to prepare the elastomer composition. The addition of an additional quantity of at least one solvent or emollient dilutes the gel composition and thereby adjusts its viscosity.

The method of preparing the polyester elastomer composition (B) as gel, suitably comprises:
i. combining a polyester elastomer with at least one solvent or emollient thereby forming a swollen polymer elastomer; and
ii. subjecting the swollen polyester elastomer to shear force thereby forming a polyester elastomer composition.

In an aspect, the polyester elastomer composition is a powder, a gel, or a paste.

In an aspect, the at least one solvent or emollient is selected from the solvents or emollients described herein.

### Synthesis of the Polyester Elastomer Composition (B) in Biobased or Naturally Derived Solvent

Polyester elastomer (B) can be synthesized in a biobased or naturally derived solvent, which requires a reaction vessel that is equipped with Nitrogen flow, heating capacity, high viscosity mixing, and the ability to distill off water.

For example, to the vessel, 1.1 equivalents of dimer acid are added, along with 1 equivalent of diglycerol and 0.75 equivalents of isostearic acid. The raw materials are followed by the addition of 10-70 wt % of a biobased or naturally derived solvent, which may be a triglyceride solvent, a mono-ester solvent, a di-ester solvent, a citrate ester solvent, an ether solvent, a carbonate solvent, a hydrocarbon solvent, a silicone solvent, and combinations thereof. After all ingredients are charged under agitation, the temperature of the mass is raised to 160° C., and water is stripped off as it forms. The temperature is held until gelation takes place and the polymer elastomer rubber is formed, which occurs between 20 and 150 hours. Thereafter, the elastomer is broken into a powder by mechanical stirring. This elastomer rubber powder is then characterized by its swell value and % crosspolymer value, which are determined by the swell test and Soxhlet extraction, described below. On average, the natural elastomer rubber is composed of 65-95% crosslinked polymer.

### Synthesis of the Polyester Elastomer Composition (B) in UV Filters (A)

In an aspect the invention relates to a composition which comprises
(A) at least one UV filter compound, and
(B) at least one crosslinked polyester component, comprising a crosslinked polyester which is reaction product of:
(i) at least one compound selected from a polycarboxylic acid, a poly-carboxylic acid ester, and combinations thereof,
(ii) at least one polyol, and
(iii) optionally one or more monofunctional component selected from
(a) a mono-carboxylic acid; and
(b) a mono-alcohol,
wherein the reaction to form the crosslinked polyester is carried out in the presence of one or more suitable, in particular, at the esterification reaction temperature liquid and stable UV filter compounds (A). It is also possible to carry out the esterification reaction in the presence of one or more UV filter compounds (A) and one or more solvents. This allows also to use solid UV filter compounds (A), which are dispersed in the solvent at the esterification reaction.

These embodiments lead to an intimate mixture of the UV filter compound (A), and the crosslinked polyester component (B), where the UV filter compound (A) can be encapsulated by the crosslinked polyester component (B), thereby providing stability and a good dispersion of the UV filter compound (A) in particular in a resulting gel.

In an aspect the invention relates therefore to a a composition which is consisting of
(A) at least one UV filter compound, and
(B) at least one crosslinked polyester component, where the esterification process to form the crosslinked polyester has been carried in the presence of the at least one UV filter compound (A), and
optionally one or more solvents, which are preferably chosen from solvents suitable as cosmetically acceptable carriers.

For example, a polyester elastomer (B) can be synthesized in an organic UV filter (A), which requires a reaction vessel that is equipped with Nitrogen flow, heating capacity, high viscosity mixing, and the ability to distill off water. To the vessel, 1.1 equivalents of dimer acid are added, along with 1 equivalent of diglycerol and 0.75 equivalents of isostearic acid, followed by 10-50 wt % of a UVB filter as the reaction solvent, which may be ethylhexylsalicylate solvent, ethylhexyl solvent, triazone solvent, homosalate solvent, isoamyl p-Methoxycinnamate solvent, menthyl anthranilate solvent, octocrylene solvent, phenylbenzimidazole sulfonic acid solvent, triethanolamine salicylate solvent, and combinations thereof. After all ingredients are charged under agitation, the temperature of the mass is raised to 160° C., and water is stripped off as it forms. The temperature is held until gelation takes place and the polymer elastomer rubber is formed, which occurs between 20 and 150 hours. Thereafter, the elastomer is broken into a powder by mechanical stirring. This elastomer rubber powder is then characterized by its swell value and % crosspolymer value, which are determined by the swell test and Soxhlet extraction, described below. Using a UVB Filter as the reaction solvent as opposed to a bio-based solvent decreases increases the % crosspolymer reached (85-95% crosspolymer).

### Characterization of the Crosslinked Polyester Components (B)

In an aspect, the solubility of polyester elastomer (B) is measured by mixing 1 gram of polyester elastomer with 100 gram of test solvent with magnetic stirring in a sealed glass container for 24 hours under room temperature (23° C.). Afterward the polyester elastomer (B) and test solvent is passed through filtration and the solid on filter is obtained and dried under 80° C. for 20 hours or dried to constant weight, optionally in vacuo. The dried solid is considered as the fraction of polyester elastomer (B) which is not soluble in the test solvent.

In an aspect, the fraction of the polyester elastomer (B) which is not soluble in the ethyl acetate by weight is greater than or equal to 40% of the total weight of the polyester elastomer (B).

In another aspect, the fraction of the polyester elastomer (B) which is not soluble in ethyl acetate is 40% to 90% of the total weight of the polyester elastomer (B). In another aspect, the fraction of the polyester elastomer (B) which is not soluble in ethyl acetate is 50% to 90% of the total weight of the polyester elastomer (B).

A sample of polyester elastomer (B) without solvent or emollient, or a sample of polyester elastomer (B) with solvent or emollient can be used. In case a sample of polyester elastomer (B) with solvent or emollient is used, the weight of the solvent or emollient in the composition is considered and subtracted from the total composition weight to determine the weight percentage of the fraction of the polyester elastomer (B) which is not soluble in the ethyl acetate.

The method of Soxhlet extraction can be used to determine the % by weight of crosslinked polyester (gel fraction) in the polyester elastomer (B). The basis for the test is to extract soluble low Mw components (and optionally present solvents or emollients) from the high Mw and crosslinked insoluble components through an extraction test. The percentage of the crosslinked polyester is defined as the ratio of the weight of insoluble residue (dried gel) to the initial weight of the polyester elastomer sample.

The gel fraction of the polyester elastomers (B), defined as

$$Gel fraction ( \% ) = 100 \times \frac{weight of dried gel ( insoluble residue of the extraction )}{total weight of the polyester elastomer used in the extraction} .$$

is determined as follows.

A sample of polyester elastomer without solvent or emollient, or a sample of polyester elastomer with solvent or emollient can be used. In case a sample of polyester elastomer with solvent or emollient is used, the weight of the solvent or emollient in the polyester elastomer is considered and subtracted from the sample weight to determine the total weight of the polyester elastomer (B) used in the extraction in the formula above. A cellulose extraction thimble is weighed. 3.0-3.5 grams of a sample of a polyester elastomer is weighed and placed in the cellulose extraction thimble. About 125-150 mL of ethyl acetate (EtOAc) are placed in a 250 mL round bottom flask. The thimble with the sample is placed into the Soxhlet extraction column. The EtOAc solution is heated to 80° C. (Reflux, bp 77° C.) and maintained at reflux for 1 hour to extract the polymer sample solubles and the optional present solvent or emollient. After 2 hours the EtOAc solution is allowed to cool to room temperature (about 23° C.). The thimble containing the polymer residue is removed from the Soxhlet apparatus. The thimble is placed in a desiccator under vacuum, a vacuum oven, or a vented oven (50° C.) to remove the residual EtOAc (24 hours). After the EtOAc has been removed, the weight of the thimble containing the dried gel is determined. The weight of the dried gel is calculated by subtraction of the weight of the cellulose extraction thimble. The gel fraction is calculated from the above equation. Any amount of solvent or emollient in the sample is subtracted from the “total weight of the polyester elastomer (B) used in the extraction”.

In an aspect, the gel fraction of the polyester elastomer (B) is greater than 20%. In an aspect, the gel fraction of the polyester elastomer (B) is greater than 40%. In an aspect, the gel fraction of the polyester elastomer (B) is greater than 50%. In an aspect, the gel fraction of the polyester elastomer is greater than 60%. In an aspect, the gel fraction of the polyester elastomer (B) is greater than 70%.

Swelling test for the polyester elastomer (B) is to determine the swelling capacity of the polyester elastomer (B) through a weight of solvent or emollient retained by the polyester elastomer. The swelling ratio (SR-sometimes called swelling value) is determined according to the equation:

$$SR = \frac{Ws - Wi}{Wi}$$

where:
Ws is the weight of the swollen polyester elastomer, and
Wi is the weight of the initial (dry polymer).

The swelling ratio of the polyester elastomer (B) is suitably determined as follows.

A sample of polyester elastomer (B) without solvent or emollient, or a sample of polyester elastomer with solvent or emollient can be used. In case a sample of polyester elastomer (B) with solvent or emollient is used, the weight of the solvent or emollient already present in the composition is considered and subtracted from the initial sample weight Wi.

The swelling procedure is carried out at ambient temperature (23° C.).

About 1.9-2.1 grams of the polyester elastomer is placed in a 25 ml beaker. In the same beaker the polyester elastomer is mixed with 24.9-25.1 grams of coco-caprylate/caprate as solvent. The polyester elastomer is allowed to disperse and absorb (swell) the solvent for 30 minutes. The weight of the filter component (such as Thermo Scientific™ Nalgene™ Rapid-Flow™ Sterile Disposable Filter Units) is determined. After the polyester elastomer has swelled, the mixture in the beaker is mixed and poured into the filter. The beaker is rinsed with about 4.9-5.1 grams of coco-caprylate/caprate solvent to complete transfer of the swelled polyester elastomer. The excess solvent in the gel mixture is allowed to pass through the filter. The filter with swollen polyester elastomer is weighed when no excess solvent is observed on its surface (which can take about 4-18 hours) to give Ws.

The swelling ratio (SR) is calculated by the equation above.

In an aspect, the swelling ratio of the elastomer (B) is from about 1 gram/gram to about 15 gram/gram, from about 1 gram/gram to about 5 gram/gram, from about 1 gram/gram to about 4 gram/gram, from about 1 gram/gram to about 2 gram/gram. In some embodiments, the swelling value of the elastomer is about 15 gram/gram, about 14 gram/gram, about 13 gram/gram, about 12 gram/gram, about 11 gram/gram, about 10 gram/gram, about 9 gram/gram, about 8 gram/gram, about 7 gram/gram, about 6 gram/gram, 5 gram/gram, about 4.8 gram/gram, about 4.6 gram/gram, about 4.4 gram/gram, about 4.2 gram/gram, about 4 gram/gram, about 3.8 gram/gram, about 3.6 gram/gram, about 3.4 gram/gram, about 3.2 gram/gram, about 3 gram/gram, about 2 gram/gram, or about 1 gram/gram.

### Swelling of Polyester Elastomer (B) in Biobased or Naturally Derived Solvent

After synthesis, the polyester elastomer (B) is evaluated by determining its swell value in various types of solvents such as triglyceride solvents, mono-ester solvents, di-ester solvents, citrate ester solvents, ether solvents, carbonate solvent, hydrocarbon solvents, silicone solvents, UV filter solvents, and combinations thereof. The elastomer synthesized in a biobased or naturally derived solvent was swelled in homosalate, isododecane, squalane, dodecane, C9-12 alkane, undecane (and) tridecane, dicaprylyl ether, jojoba oil, isoamyl laurate, heptyl undecylenate, and coco-caprylate/caprate to better understand its optimal swelling solvent. It was observed that the natural elastomer rubber swells most in ester solvents and UV filter solvents, and least in hydrocarbon solvents.

### Swelling of Polyester Elastomer (B) in UV Filters

After synthesis, the polyester elastomer rubber is evaluated by determining its swell value in various types of solvents such as triglyceride solvents, mono-ester solvents, di-ester solvents, citrate ester solvents, ether solvents, carbonate solvent, hydrocarbon solvents, silicone solvents, UV filter solvents, and combinations thereof. The elastomer synthesized in a biobased or naturally derived solvent was swelled in homosalate, isododecane, squalane, dodecane, C9-12 alkane, undecane (and) tridecane, dicaprylyl ether, jojoba oil, isoamyl laurate, heptyl undecylenate, and coco-caprylate/caprate to better understand its optimal swelling solvent. It was found that the natural elastomer rubber swells most in ester solvents and UV filter solvents, and least in hydrocarbon solvents. Overall, the natural elastomer synthesized in UVB filter solvents swells to a lesser degree than that which is made in a non-UVB filter solvent, perhaps due to its higher degree of % crosspolymer present in the elastomer rubber.

### Polyester Elastomer Component (B) Used as a Gel

As mentioned before in an aspect, a polyester elastomer composition (B) is prepared by shearing the polyester elastomer with a solvent or emollient, as described herein, to form a sheared polyester elastomer gel. In another aspect, a polyester elastomer gel is prepared by combining the polyester elastomer, as described herein, with a solvent or emollient, as described herein, thereby forming a mixture and shearing the mixture.

In an aspect, the shear force is provided by any type of mixing and shearing equipment. In an aspect, the mixing and shearing equipment is batch mixer, planetary mixer, single or multiple screw extruder, dynamic or static mixer, colloid mill, homogenizer, sonolator, three roll mill, or a combination thereof.

Subjecting these polyester elastomer compositions to a shearing force produces a polyester elastomer gel (B) which is particular suitable for use in the personal care or cosmetic applications that has an improved spreadability and an improved substance or feel. The claimed cosmetic or personal care applications where this property is most desirable included, but is not limited to, can have for example the form of skin creams, facial creams, hair care products such as shampoos, mousses, and styling gels, protective creams, color cosmetics such as lipsticks, foundations, blushes, makeup, and mascara, and other cosmetic formulations, each comprising UV filter compounds (A).

In an aspect, the viscosity of the polyester elastomer (B) used in gel form is from about 10 cp to about 1,000,000 cp as measured by rheometer at a shear rate of 0.1 s-1. In an aspect, the viscosity of the gel at 25° C. is from about 30,000 cp to about 900,000 cp. In an aspect, the viscosity of the gel is about 10 cp, about 1,000 cp, about 5,000 cp, about 10,000 cp, about 15,000 cp, about 20,000 cp, about 25,000 cp, about 30,000 cp, about 35,000 cp, about 40,000 cp, about 45,000 cp, about 50,000 cp, about 55,000 cp, about 60,000 cp, about 65,000 cp, about 70,000 cp, about 75,000 cp, about 80,000 cp, about 85,000 cp, about 90,000 cp, about 95,000 cp, about 100,000 cp, about 150,000 cp, about 200,000 cp, about 250,000 cp, about 300,000 cp, about 350,000 cp, about 400,000 cp, about 450,000 cp, —40—about 500,000 cp, about 550,000 cp, about 600,000 cp, about 650,000 cp, about 700,000 cp, about 750,000 cp, about 800,000 cp, about 850,000 cp, about 900,000 cp, about 950,000 cp, or about 1,000,000 cp.

The viscosity of the polyester elastomer gel is measured by Anton Paar rheometer MCR 301, with probe PP25/S at gap 1 mm. The measuring profile is to use flow curve with shear rate 0.01-100/s under temperature 25° C. In measurement, a sample is loaded onto the rheometer stage, the probe is lowered, and the sample is allowed to equilibrate for 3 minutes, after which the test is performed. The viscosity at 10/s is reported.

In an aspect, the polyester elastomer (B) used as gel is comprised of particles of size from about 1 μm to about 500 μm as measured by a laser diffraction particle size analyzer. In an aspect, the gel is comprised of particles of size from about 20 μm to about 400 μm. In an aspect, the gel is comprised of particles of size of about 1 μm, about 5 μm, about 10 μm, about 15 μm, about 20 μm, about 25 μm, about 30 μm, about 35 μm, about 40 μm, about 45 μm, about 50 μm, about 75 μm, about 100 μm, about 125 μm, about 150 μm, about 175 μm, about 200 μm, about 225 μm, about 250 μm, about 275 μm, about 300 μm, about 325 μm, about 350 μm, about 375 μm, or about 400 μm.

In an aspect, the polyester elastomer (B) used as gel is comprised of particles of size with D10 between 10 μm to 50 μm, D50 between 20 μm to 100 μm, and D90 between 30 μm to 200 μm.

The size of particles in polyester elastomer (B) used as gel is measured by HORIBA Scientific Partica LA-960 Laser Scattering Particle Size Analyzer. The sample is prepared by blending 0.3 g of elastomer gel (B) with 15 g of coco-caprylate/caprate and mixing thoroughly at 23° C. A few drops of the diluted gel sample are transferred into the cuvette containing neat coco-caprylate/caprate while constantly stirring. Once the transmittance reaches the acceptable range, the measurement is performed at 23° C.

Particle size values of D10, D50 and D90 are reported. The parameter D10 signifies the point in the size distribution, up to and including which, 10% of the total volume of material in the sample is ‘contained’. The parameter D50 signifies the point in the size distribution, up to and including which, 50% of the total volume of material in the sample is ‘contained’. The parameter D90 signifies the point in the size distribution, up to and including which, 90% of the total volume of material in the sample is ‘contained’.

Polyester elastomer (B) as a gel according to the present invention are characterized by the oscillation amplitude as well as oscillation frequency-dependent rheology tests at 25° C. In the linear viscoelastic region within the frequency range from 0.01-100 Hz, a gel has a storage modulus G′ which is always greater than the loss modulus G″. G′ and G″ here are rheological parameters known to the person skilled in the art. The elastic or storage modulus, designated as G′, is an indicator of how elastic the material is i.e., how much mechanical energy is being stored per cycle of deformation whereas, the viscous or loss modulus, namely G″, is the measure of the lost or dissipated mechanical energy as heat and/or other form per cycle of deformation and they collectively quantify the elastic or viscous fraction of viscoelastic solids and/or liquids and are described for example in Ferry, J. D., Viscoelastic Properties of Polymers, John Wiley & Sons, Inc. New York, 1980.

The polyester elastomer (B) as gels used according to the invention have an excellent yield point which, for example, has an advantageous effect on their thickening properties and also their ability to stabilize dispersed constituents of personal care formulations. For dynamic oscillation rheology tests, for example a MCR 301 Rheometer (Anton Paar, Graz, Austria) equipped with a 25 mm parallel plate steel geometry can be used.

Polyester elastomer (B) used as gels are notable for the fact that, at a shear rate of 1 l/s and a temperature of 25° C., they have a viscosity of less than 100,000,000 cp and at the same time satisfies G′>G″; Tan-8<1 within the linear viscoelastic region demonstrating a frequency nearly invariant characteristics. The polyester gels prepared by the methods described herein are characterized by good flowability, which has an advantageous effect on their handleability and processability, but nevertheless have a pronounced yield point and therefore good thickening and stabilizing properties.

In an aspect, the storage modulus (G′) of the polyester elastomer (B) used as gel is from about 10 Pa to about 100,000 Pa as measured by rheometer within linear viscoelastic region using dynamic—42—rheology. In an aspect, the storage modulus (G′) of the gel is from about 100 Pa to about 50,000 Pa. In an aspect, the storage modulus (G′) of the gel is from about 500 Pa to about 30,000 Pa, In an aspect, the storage modulus (G′) of the gel is about 10 Pa, about 100 Pa, about 500 Pa, about 700 Pa, about 800 Pa, about 1,000 Pa, about 1,500 Pa, about 2,000 Pa, about 2,500 Pa, about 5,000 Pa, about 10,000 Pa, about 15,000 Pa, about 25,000 Pa, about 50,000 Pa, or about 100,000 Pa.

In an aspect, the loss modulus (G″) of the polyester elastomer (B) used as gel is from about 10 Pa to about 100,000 Pa as measured by rheometer within linear viscoelastic region using dynamic rheology. In an aspect, the loss modulus (G″) of the gel is from about 100 Pa to about 50,000 Pa. In an aspect, the loss modulus (G″) of the gel is from about 500 Pa to about 30,000 Pa, In an aspect, the loss modulus (G″) of the gel is about 10 Pa, about 100 Pa, about 500 Pa, about 700 Pa, about 800 Pa, about 1,000 Pa, about 1,500 Pa, about 2,000 Pa, about 2,500 Pa, about 5,000 Pa, about 10,000 Pa, about 15,000 Pa, about 25,000 Pa, about 50,000 Pa, or about 100,000 Pa.

The storage modulus G′ and loss modulus G″ of the polyester elastomer (B) used as gel are measured by Anton Paar rheometer MCR 301, with probe PP25/S at gap 1 mm. The measuring profile is to use flow curve with shear rate 0.01-100/s under temperature 25° C. The measuring profile is to use amplitude sweep with oscillatory strain 0.001-100%, with frequency 1 Hz and under temperature 25° C. In measurement sample is loaded onto the rheometer stage, the probe is lowered and the sample is allowed to equilibrate for 3 minutes after which the amplitude sweep test is performed. The LVR region is determined and the respective G′ value is reported.

In an aspect, the polyester elastomer composition is prepared using the methods described herein.

In an aspect, the polyester elastomers described herein are produced using the principles of green chemistry. In an aspect, the polyester elastomers described herein are produced by a simple, efficient environmentally friendly process, with no toxic raw materials used, and no toxic side products generated.

In an aspect, the polyester elastomer gels described herein are produced using the principles of green chemistry.

In an aspect, the polyester elastomer gels described herein are produced by a simple, efficient environmentally friendly process, with no toxic raw materials used, and no toxic side products generated.

### Cosmetic or Personal Care Formulations

The present disclosure relates to cosmetic, i.e. personal care formulations comprising at least one UV filter compound (A), at least one crosslinked polyester component (B), optionally at least one cosmetically acceptable carrier (C) each as described above, and optionally one or more other components commonly used in the cosmetic field, different from components (A), (B) or (C).

In some aspects, the other components commonly used in the cosmetic field (D) are selected from the group consisting of pigment, humectant, vitamin, moisturizer, conditioner, oil, suspending agent, surfactant, emulsifier, preservative, rheology modifier, pH adjustor, reducing agent, anti-oxidant, foaming agents, de-foaming agents, chelating agents, gums or thickeners, oils, waxes, a fragrances, essential oils, and combinations thereof.

In an aspect, the personal care formulation can be a personal care application, such as a deodorant, an antiperspirant, a skin cream, a facial cream, a hair shampoo, a hair conditioner, a mousse, a hair styling gel, a hair spray, a protective cream, a lipstick, a facial foundations, lushes, makeup, and mascara, a skin care lotion, a moisturizer, a facial treatment, a personal cleanser, a facial cleanser, a bath oil, a perfume, a shaving cream, a pre-shave lotion, an after-shave lotion, a cologne, a sachet, or a sunscreen.

The cosmetic compositions comprising the one or more UV filter compounds (A) are in particular sunscreens and sun-protecting formulations as described herein.

The degree of UV protection afforded by a cosmetic composition is directly related to the amount and type of UV filters contained therein. Particularly, cosmetic sunscreen compositions must provide good protection against the sun, a measure of which is the Sun Protection Factor (SPF) value, a desirable balance between UVA and UVB protection, particularly a minimum UVA protection factor, yet have satisfactory sensory perception, such as a smooth and dry touch but not greasy feel upon application.

It is known that UV radiation with wavelengths of between 280 and 400 nm permits tanning of the human epidermis and that radiation with wavelengths between 280 and 320 nm, known as UVB rays, harms the development of a natural tan. Exposure is also liable to bring about a detrimental change in the biomechanical properties of the epidermis, which is reflected by the appearance of wrinkles, leading to premature ageing of the skin.

It is also known that UVA rays with wavelengths between 320 and 400 nm penetrate more deeply into the skin than UVB rays. UVA rays cause immediate and persistent browning of the skin. Daily exposure to UVA rays, even of short duration, under normal conditions can result in damage to the collagen fibers and the elastin, which is reflected by a modification in the microrelief of the skin, the appearance of winkles and uneven pigmentation (liver spots, lack of uniformity of the complexion).

Many photoprotective compositions have been proposed to date to overcome the effects induced by UVA and/or UVB radiation. They generally contain organic or mineral screening agents, which function according to their own chemical nature and according to their own properties by absorption, reflection or scattering of the UV radiation.

Organic chemical UV filters can be broken up into UVA and UVB filters, with some of them blocking a combination of UVA and UVB filters. UVA rays are generally split between UVA1, filters which absorb UV light in the range of 340 to 400 nm and UVA II, filters which absorb UV light in the range of 320 to 340 nm. Redness and sunburn are primarily due to UVB exposure, and SPF only measures blockage/absorption of UVB rays (280 to 315 nm). A comprehensive list of organic chemical filters is included in the table below, along with their chemical structures. In addition UVC filter compounds are considered to absorb UV light in the range of 200-280 nm.

Table 1 shows examples of the UV filter compounds (A) used in the cosmetic compositions of the invention.

TABLE 1

| | | Alternate | | | UV Filter |
|---|---|---|---|---|---|
| # | INCI name | Name | Structure | | Type |
| 1 | 4-methylbenzylidene camphor | Enzacamene | 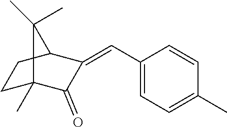 [SMILES: Cc1ccc(/C=C2\C(=O)C3(C)CCC2C3(C)C)cc1] | | UVB, UVA2 |
| 2 | Aminobenzoic acid | | 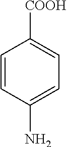 | | UVB |
| 3 | Benzophenone-3 | Oxybenzone | 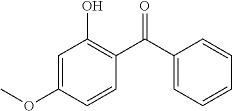 [SMILES: COc1ccc(C(=O)c2ccccc2)c(O)c1] | | UVB, UVA |
| 4 | Benzophenone-4 | Suliso- benzone | 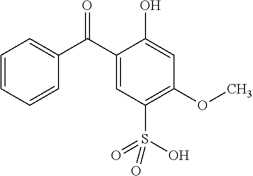 [SMILES: COc1cc(O)c(C(=O)c2ccccc2)cc1S(=O)(=O)O] | | UVB, UVA2 |
| 5 | Benzophenone-8 | Dioxy- benzone | 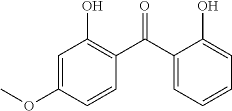 [SMILES: COc1ccc(C(=O)c2ccccc2O)c(O)c1] | | UVB, UVA2 |
| 6 | Bis-Ethylhexylphenol Methoxyphenyl Triazine | Bemotrizinol | 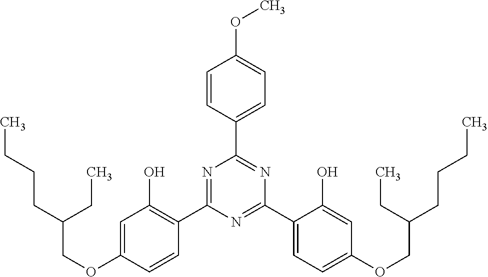 [SMILES: CCCCC(CC)COc1ccc(-c2nc(-c3ccc(OC)cc3)nc(-c3ccc(OCC(CC)CCCC)cc3O)n2)c(O)c1] | | UVB, UVA1, UVA2 |
| 7 | Butyl Methoxy- dibenzolymethane | Avobenzone | 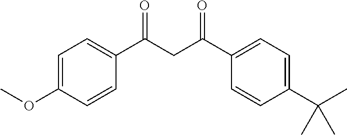 [SMILES: COc1ccc(C(=O)CC(=O)c2ccc(C(C)(C)C)cc2)cc1] | | UVA1 |
| 8 | Diethylamino Hydroxybenzoyl Hexyl Benzoate | | 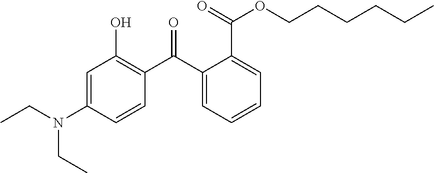 [SMILES: CCCCCCOC(=O)c1ccccc1C(=O)c1ccc(N(CC)CC)cc1O] | | UVA2 |
| 9 | Diethylhexyl Butamido Triazone | Iscotrizinol | 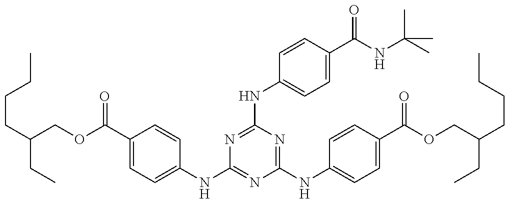 [SMILES: CCCCC(CC)COC(=O)c1ccc(Nc2nc(Nc3ccc(C(=O)NC(C)(C)C)cc3)nc(Nc3ccc(C(=O)OCC(CC)CCCC)cc3)n2)cc1] | | UVB, UVA1 |
| 10 | Disodium Phenyl Dibenzimidazole Tetrasulfonate | Bisdisulizole Disodium | 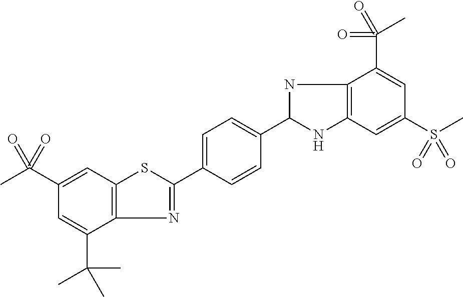 | | UVA1 |
| 11 | Drometrizole Trisiloxane | Silatrizole | 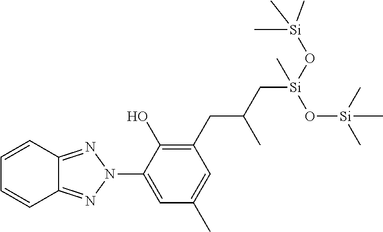 [SMILES: Cc1cc(CC(C)C[Si](C)(O[Si](C)(C)C)O[Si](C)(C)C)c(O)c(-n2nc3ccccc3n2)c1] | | UVB, UVA2 |
| 12 | Ethylhexyl Dimethyl PABA | Padimate O | 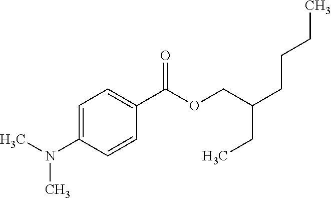 [SMILES: CCCCC(CC)COC(=O)c1ccc(N(C)C)cc1] | | UVB |
| 13 | Ethylhexyl Methoxy- cinnamate/Octyl Methoxycinnamate | Octinoxate | 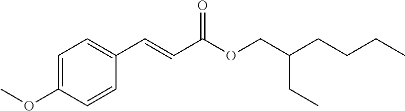 [SMILES: CCCCC(CC)COC(=O)/C=C/c1ccc(OC)cc1] | | UVB |
| 14 | Ethylhexyl salicylate | Octisalate | 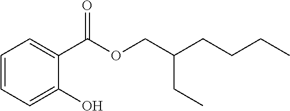 [SMILES: CCCCC(CC)COC(=O)c1ccccc1O] | | UVB |
| 15 | Ethylhexyl Triazone | Octyltriazone | 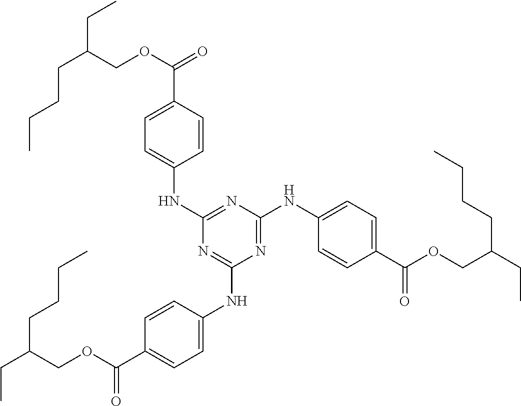 [SMILES: CCCCC(CC)COC(=O)c1ccc(Nc2nc(Nc3ccc(C(=O)OCC(CC)CCCC)cc3)nc(Nc3ccc(C(=O)OCC(CC)CCCC)cc3)n2)cc1] | | UVB, UVA2 |
| 16 | Homosalate | Homo- menthyl Salicylate | 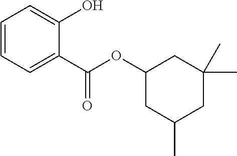 [SMILES: CC1CC(OC(=O)c2ccccc2O)CC(C)(C)C1] | | UVB |
| 17 | Isoamyl p- Methoxycinnamate | | 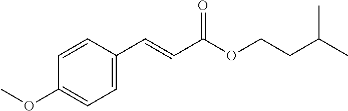 [SMILES: COc1ccc(/C=C/C(=O)OCCC(C)C)cc1] | | UVB |
| 18 | Menthyl anthranilate | Meradimate | 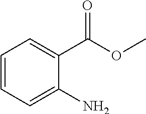 | | UVB, UVA2 |
| 19 | Methylene Bis- Benzotriazolyl Tetramethylbutylphenol (NANO) | Bisoctrizole | 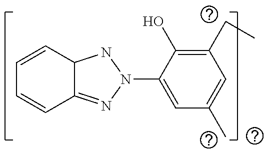 | | UVB, UVA1, UVA2 |
| 20 | Octocrylene | | 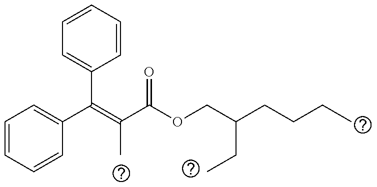 [SMILES: CCCCC(CC)COC(=O)C(C)=C(c1ccccc1)c1ccccc1] | | UVB, UVA2 |
| 21 | Phenylbenzimidazole sulfonic acid | Ensulizole | 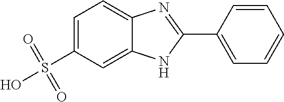 [SMILES: O=S(=O)(O)c1ccc2nc(-c3ccccc3)[nH]c2c1] | | UVB, UVA2 |
| 22 | Polysilicone-15 | | 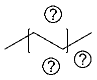 | | UVB |
| | | | 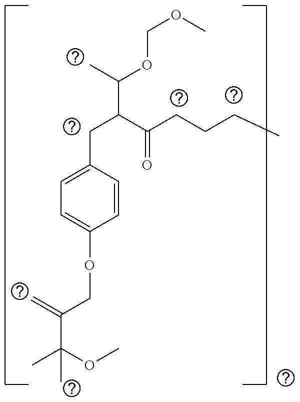 | | |
| | | | 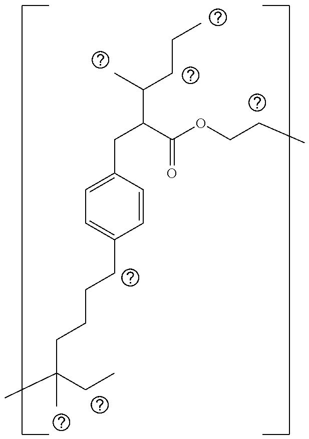 | | |
| 23 | Terepthalylidene dicamphor sulfonic acid | Ecamsule | 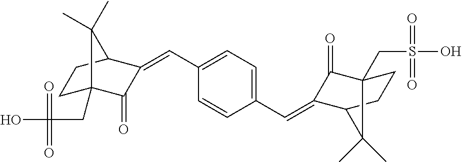 | | UVA1, UVA2 |
| 24 | Triethanolamine salicylate | TEA- Salicylate | 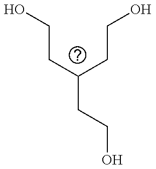 | 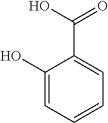 | UVB |

| | | | |
|---|---|---|---|
| 25 | Tris Biphenyl triazine (NANO) | 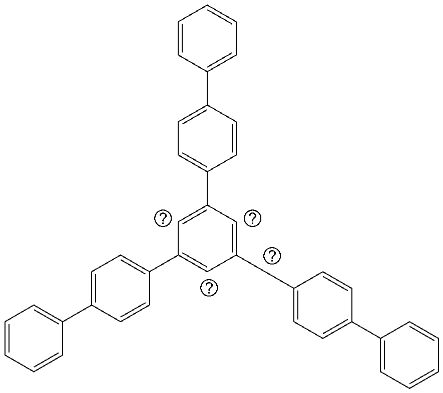 [SMILES: c1ccc(-c2ccc(Cc3cc(-c4ccc(-c5ccccc5)cc4)cc(-c4ccc(-c5ccccc5)cc4)c3)cc2)cc1] | UVB, UVA2 |
| 26 | PEG-25 PABA | 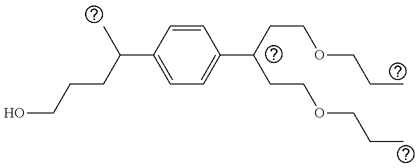 [SMILES: CCCOCCC(CCOCCC)c1ccc(C(C)CCCO)cc1] | UVB |
| 27 | Ethoxyethyl-p- Methoxycinnamate | 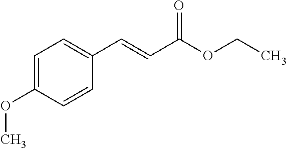 | UVB |

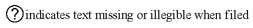

While SPF only measures protection against UVB rays, which can be seen in sunburn or damage to the skin's surface layer, UVA rays are responsible for damage to deeper tissues, and can often be the source of skin cancer. Inorganic UV filters are considered “Broad Spectrum” since they block both UVA and UVB filters from damaging skin. Inorganic UV filters are limited to types of Zinc Oxide (ZnO) and Titanium Dioxide (TiO₂), which can vary based on their chemical or physical treatment, particle size, and supplier. These mineral particles are usually used for children's skin or sensitive skin, to offer high protection of the skin against UV rays. However, these mineral screening agents have the drawback of degrading cosmetic compositions by generating a white film on the skin during application. Different types of Zinc Oxide and Titanium Dioxide are listed in Table 2 below.

TABLE 2

| INCI name | Supplier | Trade Name | Treatment |
|---|---|---|---|
| Zinc Oxide | The Innovation Company | CreaZinc Pharma C | Untreated |
| Zinc Oxide | Grillo Zinkoxid GmBH | Grillo Sun | Untreated |
| Zinc Oxide | Grillo Zinkoxid GmBH | Grillo UV ProTec | Untreated |
| Zinc Oxide | Grillo Zinkoxid GmBH | Zinc Oxide Pharma 4 | Untreated |
| Zinc Oxide | Hanil Chemical Ind. Co | KS-1 | Untreated |
| Zinc Oxide | Nanohybrid Co | NanoGard Zinc Oxide | Untreated |
| Zinc Oxide | Sino Lion USA | Nano-Zinc SL | Untreated |
| Zinc Oxide | Orient Stars | OriStar ZO | Untreated |
| Zinc Oxide | Sensient | Oxyde de Zinc | Untreated |
| | | Micropure | |
| Zinc Oxide | Sensient | UV ZNO | Untreated |
| Zinc Oxide | Universal Peserv- | Unichem ZO | Untreated |
| | A-Chem | | |
| Zinc Oxide | Zinc Corporation | USP-1 | Untreated |
| | of America | | |
| Zinc Oxide | Zinc Corporation | USP-2 | Untreated |
| | of America | | |
| Zinc Oxide | Zinc Corporation | USP-511 | Untreated |
| | of America | | |
| Zinc Oxide | BASF | Z-Cote | Untreated |
| Zinc Oxide | BASF | Z-Cote LSA UC | Untreated |
| Zinc Oxide | Symrise | Zinc Oxide Neutral | Untreated |
| Zinc Oxide | Symrise | Neo Heliopan | Untreated |
| | | ZnO 40 Pure | |
| Zinc Oxide | Jedwards International, | Zinc Oxide - USP | Untreated |
| | Inc | | |
| Zinc Oxide | Making Cosmetics | Zinc Oxide, | Untreated |
| | | Micronized | |
| Zinc Oxide | Making Cosmetics | Zinc Oxide, USP | Untreated |
| Zinc Oxide | EverCare | ZanoM | Untreated |
| Zinc Oxide | Croda | Solaveil MZP3 | Untreated |
| Zinc Oxide (and) | Symrise | Neo Heliopan | Treated with |
| Triethoxycaprylylsilane | | ZnO 40 | Triethoxycaprylylsilane |
| Zinc Oxide | KOBO | ZnO—C | None |
| Zinc Oxide (And) | KOBO | ZnO—C-12 | Treated with |
| Isopropyl Titanium | | | Isopropyl Titanium |
| Triisostearate | | | Triisostearate |
| Zinc Oxide (And) | KOBO | ZnO—C-11S2J | Treated with |
| Triethoxycaprylylsilane | | | Triethoxycaprylylsilane |
| Zinc Oxide (And) | KOBO | ZnO—C-NJE3 | Treated with Jojoba |
| Jojoba Esters | | | Esters |
| Zinc Oxide (And) | KOBO | ZNO—C-NJEP3 | Treated with Jojoba |
| Jojoba Esters (And) | | | Esters, Blended with |
| Polyhydroxystearic | | | Polyhydroxystearic |
| Acid | | | Acid |
| Zinc Oxide (And) | KOBO | ZNO—C-ASG3J | Treated with |
| Stearoyl Glutamic | | | Glutamic Acid |
| Acid | | | |
| Zinc Oxide (And) | KOBO | ZNO—C-ASGP4 | Treated with |
| Stearoyl Glutamic | | | Glutamic Acid, |
| Acid (And) | | | Blended with |
| Polyhydroxystearic | | | Polyhydroxystearic |
| Acid | | | Acid |
| Zinc Oxide (And) | KOBO | ZNO—C-NOE4 | Treated with |
| Hydrogenated | | | Hydrogenated Olive |
| Olive Oil Stearyl | | | Oil Stearyl Esters |
| Esters | | | |
| Zinc Oxide (And) | KOBO | ZNO—C-DMC2 | Treated with |
| Hydrogen | | | Hydrogen |
| Dimethicone | | | Dimethicone |
| Zinc Oxide (And) | KOBO | ZNO—C-DS4 | Treated with |
| Dimethicone | | | Dimethicone |
| Zinc Oxide (and) | Vizor Sun | Super Zinc | Treated with |
| Polyhydroxystearic | | Natural | Polyhydroxystearic |
| Acid | | | Acid |
| Zinc Oxide (and) | Vizor Sun | Super Zinc | Treated with |
| Polyhydroxystearic | | Sheer Natural | Polyhydroxystearic |
| Acid | | | Acid |
| Zinc Oxide (And) | Vizor Sun | Super Zinc 1000 | Treated with |
| Triethoxycaprylylsilane | | | Triethoxycaprylylsilane, |
| (and) Ethyl | | | Blended with |
| Ferulate | | | Ethyl Ferulate |
| Zinc Oxide (And) | Vizor Sun | Super Zinc | Treated with |
| Triethoxycaprylylsilane | | Sheer 1000 | Triethoxycaprylylsilane, |
| (and) Ethyl | | | Blended with |
| Ferulate | | | Ethyl Ferulate |
| Zinc Oxide (and) | Sunjin Beauty | SUNZnO-NAS | Treated with |
| Triethoxycaprylylsilane | Science | | Triethoxycaprylylsilane |
| Zinc Oxide (and) | Sunjin Beauty | SUNZnO-AS | Treated with |
| Triethoxycaprylylsilane | Science | | Triethoxycaprylylsilane |
| Zinc Oxide (and) | Sunjin Beauty | SUNZnO-200AS | Treated with |
| Triethoxycaprylylsilane | Science | | Triethoxycaprylylsilane, |
| (and) Cetyl | | | Blended with |
| Alcohol | | | Cetyl Alcohol |
| Zinc Oxide (and) | Sunjin Beauty | SUNZnO- | Treated with Stearic |
| Stearic Acid | Science | OLEO200SA | Acid |
| Zinc Oxide (and) | Sunjin Beauty | SUNZnO-SA | Treated with Stearic |
| Stearic Acid | Science | | Acid |
| Zinc Oxide (and) | Croda | Solaveil MZP8 | Treated with Stearic |
| Stearic Acid | | | Acid |
| Zinc Oxide (and) | Croda | Solaveil MZP7 | Treated with |
| Triethoxycaprylylsilane | | | Triethoxycaprylylsilane |
| Titanium Dioxide | Sensient | UVR TiO2 | Untreated |
| Titanium Dioxide (and) | Sensient | UVR TiO2 AS | Treated with |
| Triethoxycaprylylsilane | | | Triethoxycaprylylsilane |
| Titanium Dioxide | Symrise | Neo Heliopan ® TiO | Treated with |
| (and) Silica | | | Silica |
| Titanium Dioxide | Sunjin Beauty | MT-100SJTV | Treated with |
| (and) Aluminum | Science | | Aluminum |
| Hydroxide (and) | | | Hydroxide, Blended |
| Stearic Acid | | | with Stearic Acid |
| Titanium Dioxide | Sunjin Beauty | TX-85 | Treated with Silica, |
| (and) Silica (and) | Science | | Blended with |
| Dimethicone | | | Dimethicone |
| Titanium Dioxide | Sunjin Beauty | TX-85AS | Treated with Silica, |
| (and) Silica (and) | Science | | Blended with |
| Dimethicone | | | Dimethicone |
| Titanium Dioxide | Sunjin Beauty | TA-90 | Treated with |
| (and) Alumina | Science | | Alumina, Blended |
| (and) Stearic Acid | | | with Stearic Acid |
| Silica (and) Stearic | Sunjin Beauty | T-80SA | Treated with Silica, |
| Acid (and) | Science | | Blended with |
| Titanium Dioxide | | | Stearic Acid |
| Lauroyl Lysine | Sunjin Beauty | T-80LL | Treated with Silica, |
| (and) Silica (and) | Science | | Blended with |
| Titanium Dioxide | | | Lauroyl Lysine |
| Silica (and) | Sunjin Beauty | T-80AS | Treated with Silica, |
| Titanium Dioxide (and) | Science | | Blended with |
| Triethoxycaprylylsilane | | | Triethoxycaprylylsilane |

Suitable UV filter compounds (A) are also listed in the EU. Allowed UV Filters: Annex VI, Regulation 1223/2009/EC on Cosmetic Products, as amended by Regulation (EU) 2024/996, OJ L of 4 Apr. 2024 (https://echa.europa.cu/de/cosmetics-uv-filters).

The product Tinosorb S is preferably not used as UV filter compound (A).

In an aspect the cosmetic compositions can comprise:
0.1 to 85% by weight, preferably from 1 to 70% by weight, and more preferably from 2 to 60% by weight of one or more UV filter compounds (A),
1.0 to 80% by weight of one or more crosslinked polyester components (B),
0 to 60%, preferably 1.0 to 60% by weight of one or more cosmetically acceptable carriers (C),
0 to 60% by weight of one or more other components commonly used in the cosmetic field (D),
each based on the total weight of the composition.

In an aspect the cosmetic compositions consist of only (A), (B) and optionally (C), and preferably such compositions can comprise:
0.1 to 85% by weight, preferably from 1 to 70% by weight, and more preferably from 2 to 60% by weight of one or more UV filter compounds (A),
1.0 to 80% by weight of one or more crosslinked polyester components (B),
0 to 60% by weight of one or more cosmetically acceptable carriers (C),
each based on the total weight of the composition.
   Benefits of Using the Crosslinked Polyester Component (B), in Particular, when Used as a Gel in One or More Solvents or Emollients as Described Herein, in Sunscreens and Sun-Protecting Formulations

### Sun Protection Factor (SPF)

Skin changes with age and these changes are often most noticeable through the presence of wrinkles, dryness, decrease in fat, age spots, and sagging. The surface of the skin is known as the outer part epidermis, which thins out as people age, and the body's ability to repair skin decreases with age. Sagging of skin is due to a lower production of collagen and elastin as one gets older. Exposure to UV rays can result in harm to the skin and premature aging as well as skin cancer. Sunscreens are products that contain several ingredients including UV filters, which block, absorb, or scatter UV rays. Sunscreen can be applied on the skin in order to protect it against damage from UV radiation. UVA rays (320 to 400 nm) can contribute to advancing skin aging as well as skin cancer, and UVB rays (280 to 315 nm) can result in sunburn.

In addition to sunscreens, many beauty and personal care formulations such as foundations, primers, and moisturizers are SPF-rated, which is an important feature for consumers. When added to sunscreens and other SPF-rated personal care formulations with UV filters, the natural elastomer gel increased the SPF rating of the formulation. The UV filters in such formulations were selected from avobenzone, ethylhexyl salicylate, octocrylene, homosalate, ethyl dimethyl PABA, ethylhexyl methoxycinnamate, isoamyl p-Methoxycinnamate, menthyl anthranilate, polysilicone-15, terepthalyidene dicamphor sulfonic acid, triethanolamine salicylate, mineral UV filters, or combinations thereof. Sunscreens which contained UV filters, solvent, and the natural elastomer gel (B) show an increase in SPF compared to sunscreens without natural elastomer gel (B) and with a silicone-based elastomer gel.

### Reduced Whitening Effect of Mineral UV Filters

While SPF only measures protection against UVB rays, which can be seen in sunburn or damage to the skin's surface layer, UVA rays are responsible for damage to deeper tissues, and can often be the source of skin cancer. Inorganic UV filters are considered “Broad Spectrum” since they block both UVA and UVB filters from damaging skin. Mineral UV filters are usually used for children and sensitive skin, to offer high protection against UV rays. However, these mineral UV screening agents often come with the drawback of degrading cosmetic compositions by generating a white film on the skin during application.

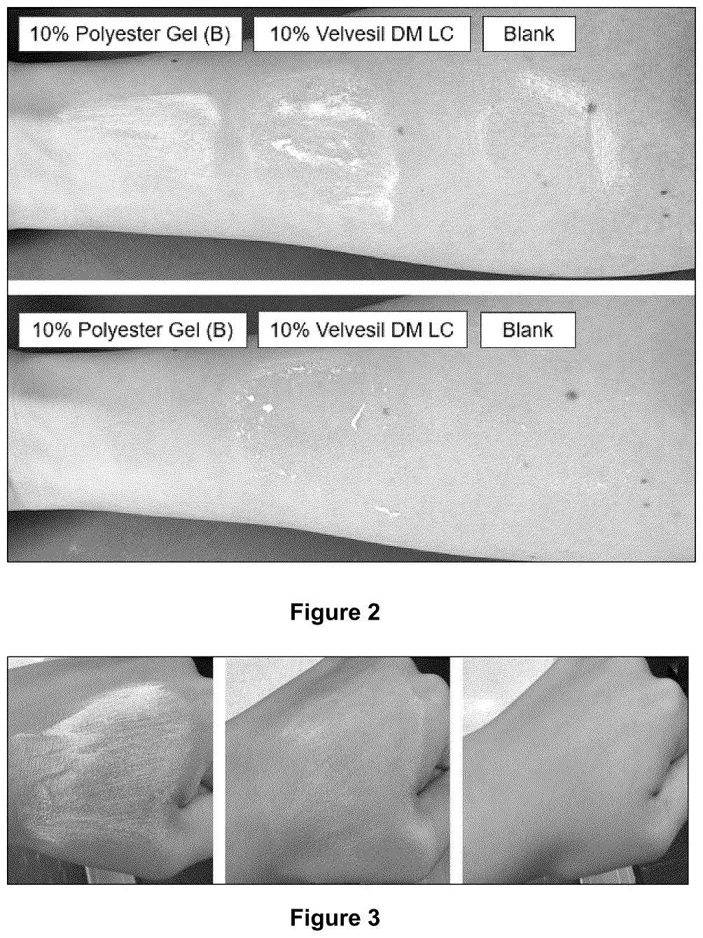

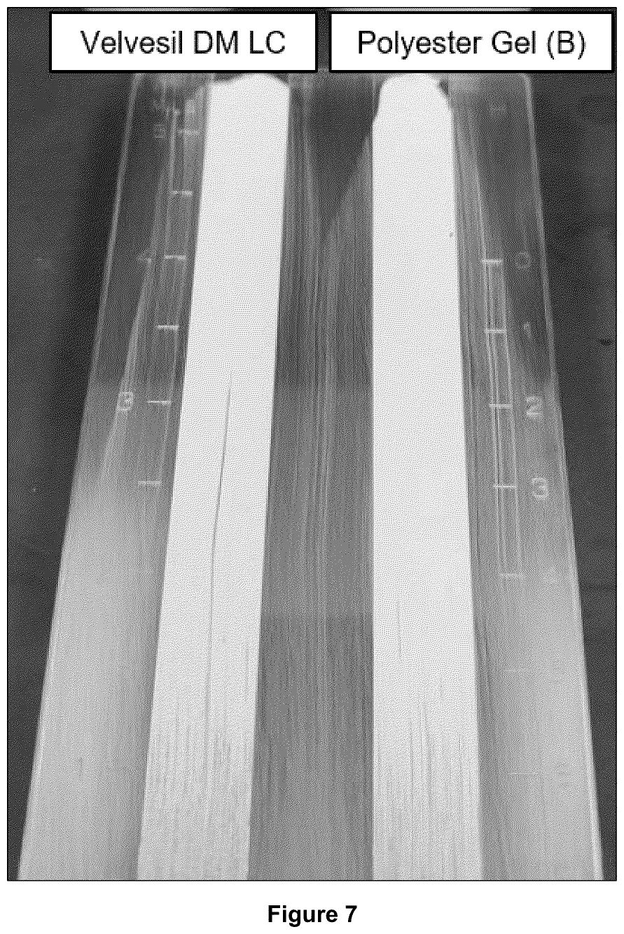

It is observed that the natural elastomer gel facilitates better dispersion of mineral UV filters, such as Zinc Oxide and Titanium Dioxide in formulations. This increased dispersion results in a reduced whitening effect of the inorganic UV filters. FIG. 2 shows sunscreens that were formulated with polyester gel (B), silicone elastomer gel Velvesil DM LC and a “blank” formulation without elastomer, and then the formulations were spread on human skin to evaluate mineral dispersion and whiteness upon application. Rub-in time, application diameter, and mass of sunscreen formulations were standardized across samples. As shown in FIG. 2, the formulation with polyester gel (B) rubs into skin easily, while the formulations with Velvesil DM LC and without any elastomer gel rub off the skin upon application. In addition, these formulations were evaluated with the Hegman gauge (FIG. 7), and the sunscreen with polyester gel showed the most even dispersion and least whiteness upon application, followed by the sunscreen with Velvesil DM LC, and finally the sunscreen without any elastomer. The results are consistent across formulations that polyester gel (B) reduces the whitening effect of mineral UV filters. FIG. 3 demonstrates the high level of absorption of a sunscreen formulation with polyester gel (B), which leaves behind no white casting on the skin after 35-40 rubs.

### Stability

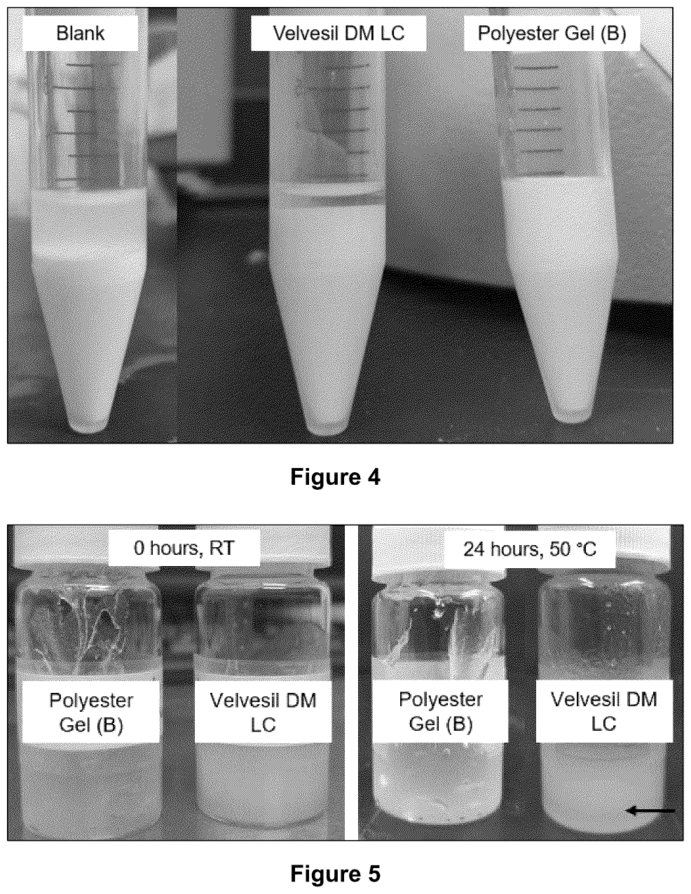

Incompatible ingredients, long exposure to sunlight or heat, and/or increased water content, as well as other factors in chemical sunscreens can lead to unstable formulations, and can cause separation over time. When a sunscreen separates, the SPF protection is no longer uniform. The separated ingredients may not be evenly distributed on the skin, leading to areas with inadequate sun protection and potentially increasing the risk of sunburn and skin damage. Thus, the stability of sunscreen formulations is critical to ensure effective SPF products, and a long shelf-life. It was observed that the crosslinked polyester component (B) in particular, when used as a gel in one or more solvents or emollients as described herein helps to stabilize sunscreen formulations. For instance, both anhydrous (Example 17) and emulsion-based sunscreen formulations (Example 21) with a polyester gel (B) were prepared and compared to the same formulations with Velvesil DM LC. Both the anhydrous and emulsion-based formulations with a polyester gel (B) were more stable after centrifugation and elevated temperature exposure, as seen in FIGS. 4 and 5. Thus, crosslinked polyester component (B), in particular, when used as a gel in one or more solvents or emollients as described herein provides a stabilizing effect to certain sunscreen formulations.

Compatibility with Formulations

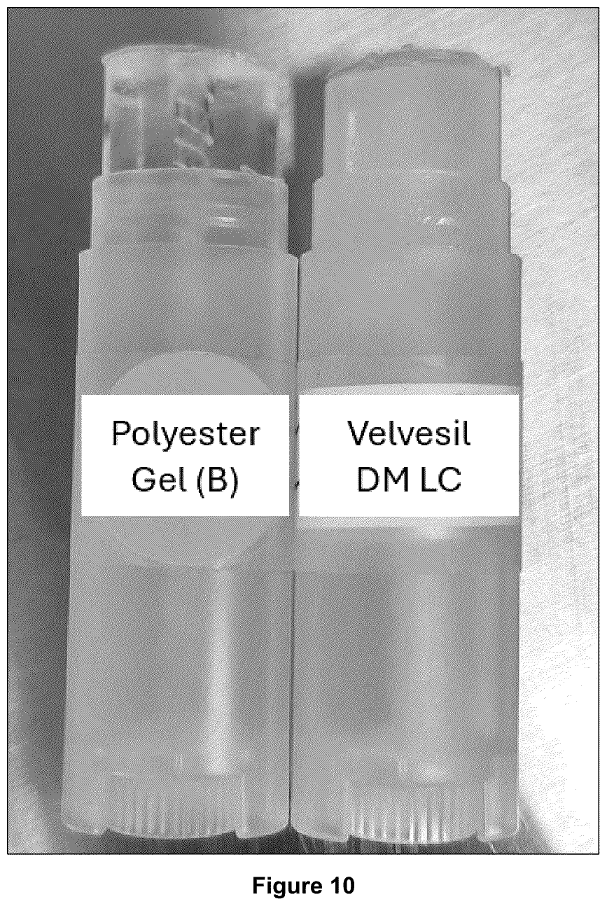

Sunscreens are available in the form of lotions, gel, spray, sticks, cream, serum, and powder, among others. Accessibility of sunscreens in different forms leads to increased consumer use and consistency, depending on preference and to which part of the body it is being applied. Sunscreen sticks are gaining popularity due to their ease of use, especially for facial applications. One challenge associated with formulating sunscreens is compatibility between active ingredients, such as UV filters, with other ingredients in the formulation. The crosslinked polyester component (B) in particular, when used as a gel in one or more solvents or emollients as described herein, provides simplicity in formulation, as it is easily incorporated into sunscreen formulations. For instance, a sunscreen stick was formulated with a polyester gel (B) and the formulation hardened and was transparent, providing a clear, spreadable and durable sunscreen stick, as seen in FIG. 10. However, when this formulation was prepared with silicone elastomer gel Velvesil DM LC, it formed a softened sunscreen stick, which collapsed upon skin application, and it remained a cloudy, opaque formulation (FIG. 10).

In addition, compatibility studies were conducted on mixtures of the crosslinked polyester component (B) in particular, when used as a gel in one or more solvents or emollients as described herein, and UV filters selected from the following: avobenzone, ethylhexyl salicylate, octocrylene, homosalate, ethyl dimethyl PABA, ethylhexyl methoxycinnamate, isoamyl p-Methoxycinnamate, menthyl anthranilate, polysilicone-15, terepthalyidene dicamphor sulfonic acid, and triethanolamine salicylate, mineral UV filters, or combinations thereof. Based on these compatibility studies, the crosslinked polyester component (B) in particular, when used as a gel in one or more solvents or emollients as described herein, mixes easily to form a blend with UV filters and does not show separation after 1-2 weeks as shown in the following table 3, which shows the result of compatibility tests of a polyester gel (B) with various UV filters at initial, 24 hour, and 1 week time points:

TABLE 3

| | | Compatible? | | |
|---|---|---|---|---|
| Component 1 | Component 2 | (Y/N) | 24 h | 1 week |
| Polyester Gel (B) | Isoamyl p- | Y | Y | Y |
| | Methoxycinnamate | | | |
| Polyester Gel (B) | Octocrylene | Y | Y | Y |
| Polyester Gel (B) | Ethylhexyl | Y | Y | Y |
| | Salicylate | | | |
| Polyester Gel (B) | Homosalate | Y | Y | Y |
| Polyester Gel (B) | Ethylhexyl | Y | Y | Y |
| | Methoxycinnamate | | | |
| Polyester Gel (B) | Tris-Biphenyl | Y | Y | Y |
| | Triazine (nano) | | | |
| Polyester Gel (B) | Octyldodecanol | Y | Y | Y |
| | (Eutanol G) | | | |
| Polyester Gel (B) | C12-15 Alkyl | Y | Y | Y |
| | Benzoate | | | |
| Polyester Gel (B) | Dibutyl | Y | Y | Y |
| | Adipate | | | |
| Polyester Gel (B) | Ethylhexyl | Y | Y | Y |
| | Methoxycinnamate | | | |
| Polyester Gel (B) | Ethylhexyl | Y | Y | Y |
| | Salicylate | | | |
| Polyester Gel (B) | Caprylic/Capric | Y | Y | Y |
| | Triglycerides | | | |
| Polyester Gel (B) | Coco-Caprylate/ | Y | Y | Y |
| | Caprate | | | |
| | (Cetiol LC) | | | |
| Polyester Gel (B) | Isononyl | Y | Y | Y |
| | Isononanoate | | | |
| Polyester Gel (B) | PPG15 Stearyl | Y | Y | Y |
| | Ether | | | |
| | (Cetiol E) | | | |
| Polyester Gel (B) | Heptyl | Y | Y | Y |
| | Undecylenate | | | |
| Polyester Gel (B) | Glycerin | Y | Y | Y |

Overall, this high compatibility of a polyester gel (B) with various UV filters allows for the effective formulation of different types of SPF formulations, without stability issues.

### Dispersion and Suspension of Mineral UV Filters

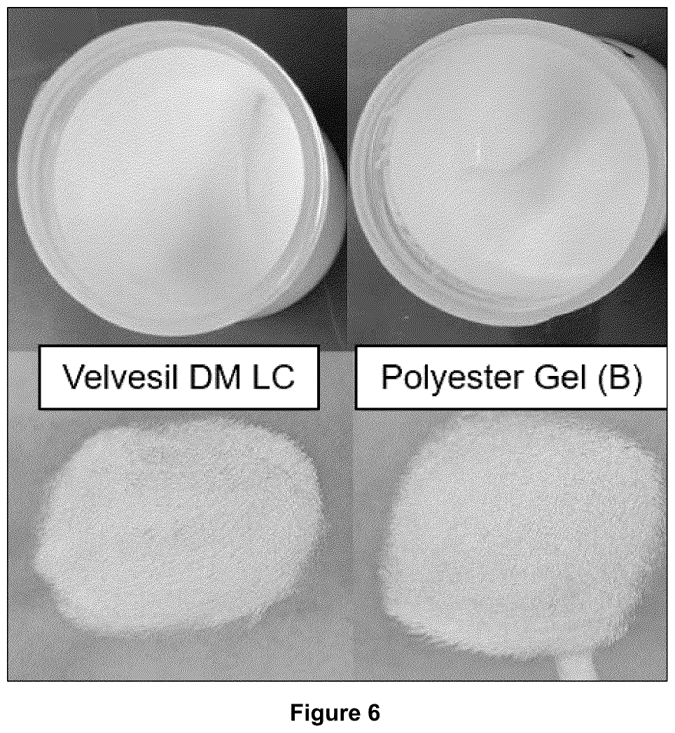

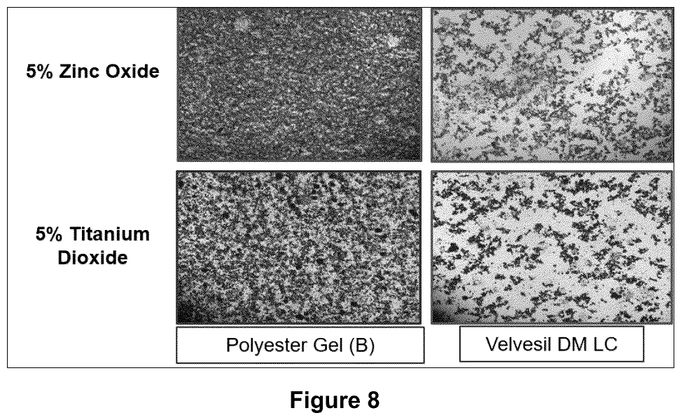

In formulations containing mineral UV filters, homogeneous dispersion and suspension of mineral filters is critical to the stability and efficacy of the formulation. It also contributes to the color uniformity of products containing mineral UV filters. Thus, it is important for mineral UV filters to be evenly dispersed into formulations such as sunscreens, and other personal care formulations with SPF including facial moisturizers, tinted moisturizers, foundations, BB creams, lip balms, serums, and others. The structure and particle size of a natural elastomer gel facilitates thorough mineral UV filter dispersion in formulations without agglomeration of the minerals, thus making it possible to incorporate minerals into a range of formulations without the need for processes or specialized equipment. The crosslinked polyester component (B) in particular, when used as a gel in one or more solvents or emollients as described herein, allows proper dispersion and suspension of mineral UV filters. In FIG. 6, the white color of the formulation with a polyester gel (B) is indicative of better formulation compatibility and pigment dispersion compared to the formulations with Velvesil DM LC. In addition, FIG. 8 shows SEM imaging of mineral UV filter formulations with a polyester gel (B) and a silicone elastomer gel. The dark/black regions shown in the images are the minerals, and the lighter regions are media without minerals. As seen by the imaging, the formulation with polyester gel (B) improves the dispersion of mineral UV filters compared to Velvesil DM LC.

### Sensory Benefits

Sunscreens are often oily and tacky in nature and can leave behind an oily residue on skin after application. However, the crosslinked polyester component (B), in particular, when used as a gel in one or more solvents or emollients as described herein, in sunscreen formulations has significantly improved their sensory properties, adding the feel of silky-smooth skin upon application. Due to its thickening effect in formulations, crosslinked polyester component (B) in particular, when used as a gel in one or more solvents or emollients as described herein, adds body and texture to sunscreens, providing a cushioning effect. The crosslinked polyester component (B) in particular, when used as a gel in one or more solvents or emollients as described herein, also improves oil absorption, which minimizes the oily feel of sunscreens. In addition, formulations with the crosslinked polyester component (B) in particular, when used as a gel in one or more solvents or emollients as described herein, remain on the skin longer than formulations without the crosslinked polyester component (B) forming long-lasting films that are non-tacky.

### Optical Benefits

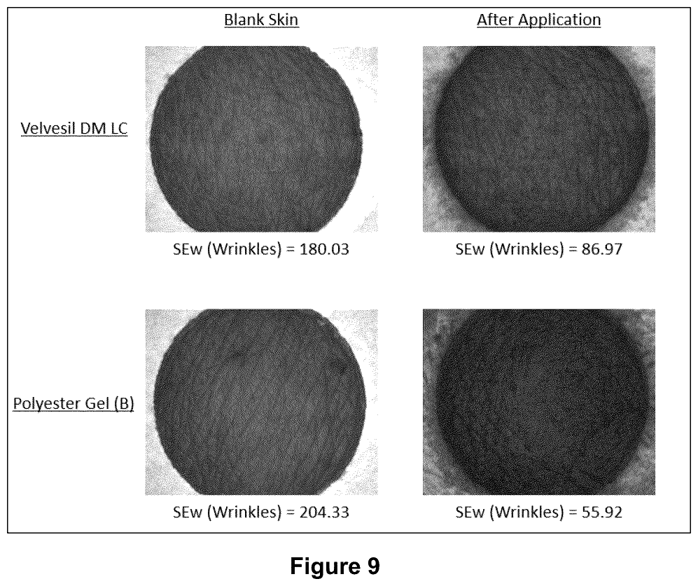

In addition to the oily feel of many sunscreens, they can often appear shiny or glossy upon application to the skin. However, the crosslinked polyester component (B) in particular, when used as a gel in one or more solvents or emollients as described herein, increases oil absorption in sunscreen applications and therefore decreases gloss of the sunscreen substantially. Gloss values of sunscreens formulated with the crosslinked polyester component (B), in particular, when used as a gel in one or more solvents or emollients as described herein, compared to sunscreens without it were much lower (measured by the BYK glossmeter). Not only does the crosslinked polyester component (B), in particular, when used as a gel in one or more solvents or emollients as described herein, provide a mattifying effect to the sunscreen formulation, but it also provides a soft-focus effect, blurring lines and improving the optical benefits of the formulation. FIG. 9 shows the difference in wrinkle-masking of sunscreens formulated with a polyester gel (B) compared to sunscreens with silicone elastomer gel Velvesil DM LC. In this figure, Visioscan images provide a quantitative wrinkle reduction value. The formulation with Velvesil DM LC provides 51.7% wrinkle reduction, while the formulation with a natural elastomer gel provides 72.6% wrinkle reduction.

### Water Resistance

One of the most important aspects to sunscreen formulations is the length of their wear. Although numerous sunscreens claim to be “waterproof,” many sunscreens are in fact resistant to oil but not to water. This is a challenge in formulating sunscreens because many chemical and physical UV filters are not inherently water-resistant. However, it was observed that the addition of crosslinked polyester component (B), in particular, when used as a gel in one or more solvents or emollients as described herein to sunscreen formulations indeed improves their water resistance.

The invention accordingly also provides a use of at least one crosslinked polyester component (B), comprising a crosslinked polyester which is reaction product of:
(i) at least one compound selected from a polycarboxylic acid, a poly-carboxylic acid ester, and combinations thereof,
(ii) at least one polyol, and
(iii) optionally one or more monofunctional component selected from
(a) a mono-carboxylic acid; and
(b) a mono-alcohol,
for boosting SPF (sun protection factor) in a cosmetic sunscreen composition, and/or
for reducing whitening in a mineral-based UV filter cosmetic sunscreen composition, and/or
for stabilizing a cosmetic sunscreen composition and/or
for thickening a cosmetic sunscreen composition and/or
for improving the compatibility of UV filter compounds (A) with other ingredients in a cosmetic sunscreen composition, and/or
for improving the sensory properties of a cosmetic sunscreen composition, and/or
for improving the optical properties of a cosmetic sunscreen composition, such as providing a mattifying effect to the sunscreen formulation, a soft-focus effect, a blurring lines effect, and/or
for improving the water resistance of a cosmetic sunscreen composition, and
a method for boosting SPF in a cosmetic sunscreen composition, and/or
for reducing whitening in a mineral-based UV filter cosmetic sunscreen composition, and/or
for stabilizing a cosmetic sunscreen composition and/or
for thickening a cosmetic sunscreen composition and/or
for improving the compatibility of UV filter compounds (A) with other ingredients in a cosmetic sunscreen composition, and/or
for improving the sensory properties of a cosmetic sunscreen composition, and/or
for improving the optical properties of a cosmetic sunscreen composition, such as providing a mattifying effect to the sunscreen formulation, a soft-focus effect, a blurring lines effect, and/or
for improving the water resistance of a cosmetic sunscreen composition comprising the step of adding at least one crosslinked polyester component (B), comprising a crosslinked polyester which is reaction product of:
(i) at least one compound selected from a polycarboxylic acid, a poly-carboxylic acid ester, and combinations thereof,
(ii) at least one polyol, and
(iii) optionally one or more monofunctional component selected from
(a) a mono-carboxylic acid; and
(b) a mono-alcohol,
to said cosmetic sunscreen composition.

### EXAMPLES

The following examples are included to demonstrate various aspects of the present disclosure. It should be appreciated by those of skill in the art that the techniques disclosed in the examples that follow represent techniques discovered by the inventors to function well in the practice of the disclosure, and thus can be considered to constitute preferred modes for its practice. However, those of skill in the art should, in light of the present disclosure, appreciate that many changes can be made in the specific examples which are disclosed and still obtain a like or similar result without departing from the spirit and scope of the disclosure.

### Example 1: Esterification to Prepare Polyester Elastomer Component (B)

In a suitable vessel equipped with agitation, heat, and an ability to distill off water, 130 g dimer acid was added along with 190 g hydrogenated castor oil. Next 50 g squalane was added as solvent. After all ingredients were charged under agitation, the temperature of the mass was raised to 180° C., and water was stripped off as formed. The temperature was held until gelation took place and polymer elastomer was formed. Thereafter the elastomer was broken into a powder by mechanical stirring.

### Example 2: Esterification to Prepare Polyester Elastomer Component (B)

In a suitable vessel equipped with agitation, heat, and an ability to distill off water, 200 g dimer acid was added along with 35 g diglycerol and 30 g oleic acid. Next 30 g squalane was added as solvent. After all ingredients were charged under agitation, the temperature of the mass was raised to 160° C., and water was stripped off as formed. The temperature was held until gelation took place and polymer elastomer was formed. Thereafter the elastomer was broken into a powder by mechanical stirring.

### Example 3: Esterification to Prepare Polyester Elastomer Component (B)

In a suitable vessel equipped with agitation, heat, and an ability to distill off water, 130 g hydrogenated dimer acid was added along with 190 g hydrogenated castor oil. Next 75 g squalane was added as solvent. After all ingredients were charged under agitation, the temperature of the mass was raised to 180° C., and water was stripped off as formed. The temperature was held until gelation took place and polymer elastomer was formed. Thereafter the elastomer was broken into a powder by mechanical stirring.

### Example 4: Esterification to Prepare Polyester Elastomer Component (B)

In a suitable vessel equipped with agitation, heat, and an ability to distill off water, 200 g dimer acid was added along with 35 g diglycerol and 30 g isostearic acid. After all ingredients were charged under agitation, the temperature of the mass was raised to 160° C., and water was stripped off as formed. The temperature was held until gelation took place and polymer elastomer was formed. Thereafter the elastomer was broken into a powder by mechanical stirring.

### Example 5: Esterification to Prepare Polyester Elastomer Component (B) in the Presence of a UV Filter Compound (A)

In a suitable vessel equipped with agitation, heat, and an ability to distill off water, 190 g dimer acid was added along with 40 g diglycerol and 35 g isostearic acid. Next 66 g homosalate was added as solvent and UV filter compound (A). After all ingredients were charged under agitation, the temperature of the mass was raised to 140° C., and water was stripped off as formed. The temperature was held until gelation took place and polymer elastomer was formed. Thereafter the elastomer was broken into a powder by mechanical stirring.

### Example 6: Esterification to Prepare Polyester Elastomer Component (B) in the Presence of a UV Filter Compound (A)

In a suitable vessel equipped with agitation, heat, and an ability to distill off water, 150 g dimer acid was added along with 39 g diglycerol and 5 g isostearic acid. Next 159 g homosalate was added as solvent and UV filter compound (A). After all ingredients were charged under agitation, the temperature of the mass was raised to 140° C., and water was stripped off as formed. The temperature was held until gelation took place and polymer elastomer was formed. Thereafter the elastomer was broken into a powder by mechanical stirring.

### Example 7: Esterification to Prepare Polyester Elastomer Component (B) in the Presence of a UV Filter Compound (A)

In a suitable vessel equipped with agitation, heat, and an ability to distill off water, 140 g dimer acid was added along with 180 g hydrogenated castor oil. Next 80 g homosalate was added as solvent and UV filter compound (A). After all ingredients were charged under agitation, the temperature of the mass was raised to 180° C., and water was stripped off as formed. The temperature was held until gelation took place and polymer elastomer was formed. Thereafter the elastomer was broken into a powder by mechanical stirring.

### Example 8: Preparation of Polyester Gel (B) from Polyester Elastomer

After an elastomer powder was produced, such as that made in Example 4, which did not separate upon standing, 32.5 g coco-caprylate/caprate was added to 175 g elastomer. After the elastomer and solvent mixture sit and swell for 1 hour, it was milled with a 3-roll mill, and then diluted with 300 g coco-caprylate/caprate. The milled elastomer solvent mixture was then homogenized in the reactor vessel to produce a creamy, translucent gel of very smooth consistency, suitable for use in personal care formulations.

### Example 9: Preparation of Polyester Gel (B) from Polyester Elastomer

After an elastomer powder was produced, such as that made in Example 3, which did not separate upon standing, 330 g coco-caprylate/caprate was added to 175 g elastomer. After the elastomer and solvent mixture sit and swell for 1 hour, it was mixed via homogenizer to produce a creamy, translucent gel of very smooth consistency, suitable for use in personal care formulations.

### Example 10: Preparation of Polyester Gel (B) from Polyester Elastomer

After an elastomer powder was produced, such as that made in Example 1, which did not separate upon standing, 20 g homosalate was added to 175 g elastomer as solvent and UV filter compound (A). After the elastomer and solvent mixture sit and swell for 1 hour, it was milled with a 3-roll mill, and then diluted with 300 g homosalate as solvent and UV filter compound (A). The milled elastomer solvent mixture was then homogenized in the reactor vessel to produce a creamy, translucent gel of very smooth consistency, suitable for use in personal care formulations.

### Example 11: Preparation of Polyester Gel (B) from Polyester Elastomer

After an elastomer powder was produced, such as that made in Example 1, which did not separate upon standing, 25 g coco-caprylate/caprate was added to 175 g elastomer. After the elastomer and solvent mixture sit and swell for 1 hour, it was milled with a 3-roll mill, and then diluted with 300 g homosalate as solvent and UV filter compound (A). The milled elastomer solvent mixture was then homogenized in the reactor vessel to produce a creamy, translucent gel of very smooth consistency, suitable for use in personal care formulations.

### Example 12: Preparation of Polyester Gel (B) from Polyester Elastomer

After an elastomer was produced, such as that made in Example 1, which did not separate upon standing, 30 g ethylhexyl salicylate was added to 175 g elastomer. After the elastomer and solvent mixture sit and swell for 1 hour, it was milled with a 3-roll mill, and then diluted with 300 g ethylhexyl salicylate. The milled elastomer solvent mixture was then homogenized in the reactor vessel to produce a creamy, translucent gel of very smooth consistency, suitable for use in personal care formulations.

### Example 13: Preparation of Polyester Gel (B) from Polyester Elastomer

After an elastomer powder was produced, such as that made in Example 1, which did not separate upon standing, 40 g coco-caprylate/caprate was added to 175 g elastomer. After the elastomer and solvent mixture sit and swell for 1 hour, it was milled with a 3-roll mill, and then diluted with 290 g ethylhexyl salicylate. The milled elastomer solvent mixture was then homogenized in the reactor vessel to produce a creamy, translucent gel of very smooth consistency, suitable for use in personal care formulations.

### Example 14: Preparation of Polyester Gel (B) from Polyester Elastomer

After an elastomer powder was produced, such as that made in Example 1, which did not separate upon standing, 25 g octocrylene was added to 175 g elastomer. After the elastomer and solvent mixture sit and swell for 1 hour, it was milled with a 3-roll mill, and then diluted with 300 g octocrylene. The milled elastomer solvent mixture was then homogenized in the reactor vessel to produce a creamy, translucent gel of very smooth consistency, suitable for use in personal care formulations.

### Example 15: Preparation of Polyester Gel (B) from Polyester Elastomer

After an elastomer powder was produced, such as that made in Example 1, which did not separate upon standing, 40 g coco-caprylate/caprate was added to 175 g elastomer. After the elastomer and solvent mixture sit and swell for 1 hour, it was milled with a 3-roll mill, and then diluted with 290 g octocrylene. The milled elastomer solvent mixture was then homogenized in the reactor vessel to produce a creamy, translucent gel of very smooth consistency, suitable for use in personal care formulations.

### Example 16: Formulation of an Oil in Water Sunscreen with Chemical UV Filters (A) and Polyester Gel (B)

| Phase | INCI Name | Wt (%) |
|---|---|---|
| A | Water | Up to 100.0 |
| | Xanthan gum | 0.2 |
| | Glycerin | 0.5 |
| | Disodium EDTA | 0.1 |
| | Carbomer | 0.2 |
| | Butylene glycol | 2.0 |
| B | Sodium stearoyl glutamate | 1.0 |
| | Glyceryl Stearate | 3.0 |
| | Ethylhexyl salicylate | 5.0 |
| | (UV filter (A)) | |
| | Homosalate (UV filter (A)) | 15.0 |
| | Avobenzone (UV filter (A)) | 3.0 |
| | Octocrylene (UV filter (A)) | 10 |
| C | Polyester Gel (B) such | 5.0 |
| | as that in Example 8 | |
| D | Preservative | As needed |

Protocol for Example 16 formulation is as follows. Wet xanthan gum with glycerin and butylene glycol; add the rest of the ingredients of phase A and heat to 40° C. until homogeneous. Mix ingredients of phase B and heat to 70-80° C. until homogeneous. Add phase C to B and mix. Slowly add phase B+C to A. Homogenize the mixture 11,000 rpm for 1 minute. Cool down and add preservative.

### Example 17: Formulation with Chemical UV Filters (A) and Polyester Gel (B)

| Phase | INCI Name | Wt (%) |
|---|---|---|
| A | Avobenzone (UV filter (A)) | 3.0 |
| | Ethylhexyl Salicylate | 5.0 |
| | (UV filter (A)) | |
| | Octocrylene (UV filter (A)) | 10.0 |
| | Homosalate (UV filter (A)) | 10.0 |
| | Polyester-7 (and) Neopentyl | 5.0 |
| | Glycol Diheptanoate | |
| B | Polyester Gel (B) such as | 47.0 |
| | that in Example 8 | |
| | Isoamyl Laurate | 10.0 |
| | Coco-Caprylate/Caprate | 5.0 |
| | Caprylic/Capric Triglyceride | 5.0 |
| | (and) Castor Oil/PDI | |
| | Copolymer | |

Protocol for Example 17 is as follows. Combine all of Phase A into a beaker and heat and mix until homogeneous. Remove from heat and add to Phase B. Homogenize phases until homogeneous.

### Example 18: Formulation with Chemical UV Filters (A) and Polyester Gel (B)

| Phase | INCI Name | Wt (%) |
|---|---|---|
| A | Avobenzone (UV filter (A)) | 3.0 |
| | Ethylhexyl Salicylate | 5.0 |
| | (UV filter (A)) | |
| | Octocrylene (UV filter (A)) | 10.0 |
| | Homosalate (UV filter (A)) | 10.0 |
| | Polyester-7 (and) Neopentyl | 5.0 |
| | Glycol Diheptanoate | |
| B | Polyester Gel (B) such as | 44.0 |
| | that in Example 8 | |
| | Isoamyl Laurate | 10.0 |
| | Coco-Caprylate/Caprate | 5.0 |
| | Caprylic/Capric Triglyceride | 8.0 |
| | (and) Castor Oil/PDI | |
| | Copolymer | |

Protocol for Example 18 is as follows. Combine all of Phase A into a beaker and heat and mix until homogeneous. Remove from heat and add to Phase B. Homogenize phases until homogeneous.

### Example 19: Sun Gel Formulation with Chemical UV Filters (A) and Polyester Gel (B)

| Phase | INCI Name | Wt (%) |
|---|---|---|
| A | Avobenzone (UV filter (A)) | 3.0 |
| | Ethylhexyl Salicylate | 5.0 |
| | (UV filter (A)) | |
| | Octocrylene (UV filter (A)) | 10.0 |
| | Homosalate (UV filter (A)) | 10.0 |
| | Polyester-7 (and) Neopentyl | 5.0 |
| | Glycol Diheptanoate | |
| B | Polyester Gel (B) such as | 56.0 |
| | that in Example 8 | |
| | Oryza Sativa Hull Powder | 2.0 |
| | Caprylic/Capric Triglyceride | 1.0 |
| | (and) Stearalkonium Hectorite | |
| | (and) Propylene Carbonate | |
| | Coco-Caprylate/Caprate | 8.0 |

Protocol for Example 19 is as follows. Combine all of Phase A into a beaker and heat to 75° C. and mix until homogeneous. Remove from heat and add in Phase B one by one while mixing.

### Example 20: Formulation with Chemical UV Filters (A) and Polyester Gel (B)

| Phase | INCI Name | Wt % |
|---|---|---|
| A | Avobenzone (UV filter (A)) | 3.0 |
| | Ethylhexyl Salicylate | 5.0 |
| | (UV filter (A)) | |
| | Octocrylene (UV filter (A)) | 4.0 |
| | Homosalate (UV filter (A)) | 8.0 |
| B | Polyester Gel (B) such as | 74.0 |
| | that in Example 8 | |
| | Coco-Caprylate/Caprate | 5.0 |
| | Phenoxyethanol (and) | 1.0 |
| | Ethylhexylglycerin | |

Protocol for Example 20 is as follows. Combine all of Phase A into a beaker and heat to 75° C. and mix until homogeneous. Remove from heat and add to Phase B. Homogenize phases until homogeneous.

### Example 21: O/W Mineral Day Cream Formulation

| Phase | INCI Name | Wt % |
|---|---|---|
| A | Water | 53.05 |
| | Xanthan Gum | 0.20 |
| | Glycerin | 1.20 |
| | Disodium EDTA | 0.05 |
| B | Sodium Stearoyl Glutamate | 0.50 |
| | Glyceryl Stearate | 4.00 |
| | Cetearyl Alcohol | 3.00 |
| | Prunus Amygdalus Dulcis | 2.00 |
| | (Sweet Almond) Oil | |
| | Caprylic/Capric Triglycerides | 2.00 |
| | C12-C15 Alkyl Benzoate | 10.00 |
| | Zinc Oxide (and) Polyhydroxystearic | 12.00 |
| | Acid (UV Filter (A)) | |
| | Polyester Gel (B) such as | 10.00 |
| | that in Example 8 | |
| C | Preservative | 2.00 |

Protocol for Example 21 is as follows. Mix Phase A until all xanthan gum is dissolved. Homogenize the natural oils and zinc oxide. Once homogeneous, add in the emulsifiers. Heat Phases A and B to 65 C. Add Phase B to Phase A, and homogenize. Add in the polyester gel (B) and homogenize. Adjust pH to 4.5-5.0. Add in Phase C.

### Example 22: Formulation with Mineral UV Filters (A) and Polyester Gel (B)

| | Trial #1 | Trial #2 |
|---|---|---|
| Component | (g) | (g) |
| Titanium Dioxide | 5 | 5 |
| (UV Filter (A)) | | |
| Zinc Oxide (UV Filter (A)) | 5 | 5 |
| Heptyl Undecylenate | 5 | 5 |
| Polyester Gel (B) such | 5 | 0 |
| as that in Example 8 | | |
| Velvesil DM LC | 0 | 5 |

Protocol for Example 22 is as follows. Add components to a FlackTek cup and high-speed mix for 5 minutes at 3000 rpm.

### Example 23: Formulation with Mineral UV Filters (A) and Polyester Gel (B)

| Phase | INCI Name | Wt % |
|---|---|---|
| A | Water | 31.0 |
| | Magnesium Sulfate | 1.0 |
| | Propanediol | 2.0 |
| | Preservative | 2.0 |
| B | Polyester Gel (B) such as | 10.0 |
| | that in Example 8 | |
| | Coco-Caprylate/Caprate | 24.0 |
| | Polyglyceryl-6 Polyhydroxystearate, | 5.0 |
| | Polyglyceryl-6 Polyricinoleate, | |
| | Polyglycerin-6 | |
| | Zinc Oxide (UV Filter (A)) | 25.0 |

Protocol for Example 23 is as follows. Combine all of Phase A into a beaker and slowly heat to 80° C. Combine all of Phase B and slowly heat to 80° C. and mix until homogeneous. Under homogenization, slowly add Phase A to Phase B. Slowly cool to 40° C. with mixing.

### Example 24: Swelling Values of Elastomer Components (B)

Swelling values (or ratios) of certain polyester elastomer components (B) in homosalate, octocrylene, octisalate, isoamyl p-methoxycinnamate, isododecane, squalane, dodecane, C9-12 alkane, undecane (and) tridecane, dicaprylyl ether, jojoba oil, isoamyl laurate, heptyl undecylenate, and coco-caprylate/caprate were measured as described herein. Certain polyester elastomer components (B) had the highest swelling values with UV filters, indicating a high level of compatibility, compared to other solvents. The swelling values of certain polyester elastomer components (B) rank as follows: 1) octocrylene 2) octisalate 3) isoamyl p-methoxycinnamate 4) homosalate 5) heptyl undecylenate 6) isoamyl laurate 7) coco-caprylate/caprate 8) dicaprylyl ether 9) jojoba oil 10) squalane, dodecane 11) C9-12 alkane 12) undecane (and) tridecane, isododecane.

### Example 25: Invisible Sunscreen Stick Formulation

| Phase | INCI Name | Wt (g) |
|---|---|---|
| A | Avobenzone | 3.00 |
| | Ethylhexyl Salicylate | 5.00 |
| | Octocrylene | 10.00 |
| | Homosalate | 10.00 |
| | Dibutyl Ethylhexanoyl Glutamide | 10.00 |
| | (and) Dibutyl Lauroyl Glutamide | |
| | (and) Octyldodecanol | |
| | C12-15 Alkyl Benzoate | 5.00 |
| | Polyester-7 (and) Neopentyl | 5.00 |
| | Glycol Diheptanoate | |
| B | Polyester Gel (B) such as | 10.0 |
| | that in Example 8 | |
| | Diheptyl Succinate (and) | 10.00 |
| | Capryloyl Glycerin/Sebacic | |
| | Acid Copolymer | |
| | Oryza Sativa Hull Powder | 0.80 |
| | Coco-Caprylate/Caprate | 13.20 |

Protocol for Example 25 is as follows. Combine Phase A and heat to 90° C. while mixing. Add the components from Phase B, and once dissolved, add Polyester Gel (B) and mix until homogeneous.

1. A composition comprising:
(A) at least one UV filter compound, and
(B) at least one crosslinked polyester component, wherein the at least one crosslinked polyester component comprises a crosslinked polyester which is a reaction product of:
(i) at least one compound selected from the group consisting of poly-carboxylic acids, poly-carboxylic acid esters, and combinations thereof, and
(ii) at least one polyol.

2. The composition of claim 1, wherein the crosslinked polyester of (B) is further the reaction product of:
(iii) at least one mono-carboxylic acid; or
(iii) at least one mono-carboxylic acid and (iv) one or more mono-alcohol.

3. The composition of claim 1, further comprising
(C) at least one cosmetically acceptable carrier; or
(C) at least one cosmetically acceptable carrier and (D) one or more other components commonly used in the cosmetic field; wherein the one or more other components commonly used in the cosmetic field (D) are different than (A), (B), and (C).

4. The composition of claim 1, wherein the composition is a sunscreen composition.

5. The composition of claim 1, wherein the at least one UV filter compound (A) is selected from the group consisting of UVA filter compounds, UVB filter compounds, UVC filter compounds, and combinations thereof.

6. (canceled)

7. The composition of claim 3, wherein the at least one cosmetically acceptable carrier (C) is selected from the group consisting of water, solvents, emollients, fatty acids, fatty alcohols, film formers, emulsifiers, thickeners, and combinations thereof.

8. The composition of claim 1, wherein:
the at least one compound (i) is selected from the group consisting of
(1) one or more poly-carboxylic acids of formula (I) 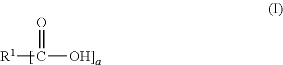
wherein
R¹ is selected from the group consisting of C2-C52 alkyl group, C2-C52 heteroalkyl group, C2-C52 alkene group, C2-C52 heteroalkene group, C3-C52 cyclic group, and C2-C52 heterocyclic group, and
a is an integer from 2 to 10,
(2) one or more poly-carboxylic acid esters of formula (II) 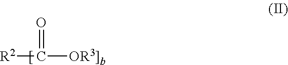
wherein
R² is C1-C22 alkyl group, C2-C22 alkylene group, or C3-C22 cyclic group;
R³ is C2-C200 alkyl group, C2-C200 heteroalkyl group, C2-C200 alkene group, C2-C200 heteroalkene group, C2-C200 alkyne group, C2-C200 heteroalkyne group, C2-C200 cyclic group, or C2-C200 heterocyclic group; and
b is an integer from 2 to 10, and
(3) combinations thereof,
the at least one polyol (ii) is a polyol of formula (III) 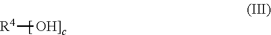
wherein
R⁴ is C2-C200 alkyl group, C2-C200 heteroalkyl group, C2-C200 alkene group, C2-C200 heteroalkene group, C2-C200 alkyne group, C2-C200 heteroalkyne group, C3-C200 cyclic group, or C2-C200 heterocyclic group; and
c is an integer from 2 to 10.

9. The composition of claim 1, wherein:
the at least one compound (i) is one or more poly-carboxylic acids selected from the group consisting of di-carboxylic acids, tri-carboxylic acids, and combinations thereof; and
the at least one polyol (ii) is selected from the group consisting of diols, triols, and combinations thereof.

10. (canceled)

11. The composition of claim 1, wherein the at least one crosslinked polyester component (B) has a gel fraction which is greater than 20%.

12. The composition of claim 1, wherein the at least one crosslinked polyester component (B) has a swelling ratio of from about 1 gram/gram to about 15 gram/gram.

13. The composition of claim 1, wherein the at least one crosslinked polyester component (B) is comprised of particles of a size from about 1 μm to about 500 μm as measured by a laser diffraction particle size analyzer.

14. The composition of claim 1, wherein the at least one crosslinked polyester component (B) is obtained by:
(a) reacting the at least one compound (i) and the at least one polyol (ii) to obtain the crosslinked polyester of (B); and
(b) subjecting the crosslinked polyester of (B) obtained in (a) shear force to obtain the cross-linked polyester component (B); or
(c) adding one or more solvents, one or more emollients, or a combination thereof to the crosslinked polyester of (B) obtained in (a), allowing the resulting mixture to swell in the one or more solvents, and subjecting the swollen mixture to shear force to obtain the at least one cross-linked polyester component (B).

15. (canceled)

16. The composition of claim 14, wherein the shear force in (b) and (c) is carried out with a batch mixer, planetary mixer, single or multiple screw extruder, dynamic or static mixer, colloid mill, homogenizer, sonolator, three roll mill, or a combination thereof.

17. (canceled)

18. The composition of claim 14, wherein the amount of the one or more solvents added in (c) is such that the resulting crosslinked polyester component (B) comprises from about 10% to about 95% weight by weight of the one or more solvents.

19. The composition of claim 1,
wherein the reaction to form the at least one crosslinked polyester component (B) is carried out in the presence of the at least one UV filter compound (A).

20. (canceled)

21. The composition of claim 3, wherein the one or more other components commonly used in the cosmetic field (D) are selected from the group consisting of pigment, humectant, vitamin, moisturizer, conditioner, oil, suspending agent, surfactant, emulsifier, preservative, rheology modifier, pH adjustor, reducing agent, anti-oxidant, foaming agents, de-foaming agents, chelating agents, gums or thickeners, oils, waxes, a fragrances, essential oils, and combinations thereof.

22. The composition of claim 1, comprising:
0.1 to 85% by weight of the at least one UV filter compound (A),
1.0 to 80% by weight of the at least one crosslinked polyester component (B),
0 to 90% by weight of at least one cosmetically acceptable carrier (C),
0 to 60% by weight of one or more other components commonly used in the cosmetic field (D),
based on the total weight of the composition.

23. (canceled)

24. A personal care formulation comprising the composition of claim 1, wherein the personal care formulation is selected from the group consisting of skin creams, facial creams, hair care products, protective creams, and color cosmetics.

25. A composition consisting of:
(A) at least one UV filter compound, and
(B) at least one crosslinked polyester component, wherein the at least one crosslinked polyester component comprises a crosslinked polyester which is a reaction product of:
(i) at least one compound selected from the group consisting of poly-carboxylic acids, poly-carboxylic acid esters, and combinations thereof, and
(ii) at least one polyol; or
(A) at least one UV filter compound;
(B) at least one crosslinked polyester component, wherein the at least one crosslinked polyester component comprises a crosslinked polyester which is a reaction product of:
(i) at least one compound selected from the group consisting of poly-carboxylic acids, poly-carboxylic acid esters, and combinations thereof, and
(ii) at least one polyol; and
(C) at least one cosmetically acceptable carrier.

26.-28. (canceled)

29. A sunscreen formulation comprising:
(A) at least one UV filter compound, and
(B) at least one crosslinked polyester component, wherein the at least one crosslinked polyester component comprises a crosslinked polyester which is a reaction product of:
(i) at least one compound selected from the group consisting of poly-carboxylic acids, poly-carboxylic acid esters, and combinations thereof, and
(ii) at least one polyol;
wherein the sunscreen formulation comprising the at least one crosslinked polymer component (B) provides a benefit compared to a sunscreen formulation without the at least one crosslinked polymer component (B), wherein the benefit is selected from the group consisting of:
a reduction in whitening effect when the at least one UV filter compound (A) is a mineral-based UV filter;
an increase in stability;
an increase in thickness;
an increase in compatibility with other ingredients commonly used in a sunscreen formulation;
an increase in smoothness;
an increase in optical properties;
an increase in water resistance; and
combinations thereof.

30. The composition of claim 2, wherein:
the at least one mono-carboxylic acid (iii) is a mono-carboxylic acid of formula (IV) 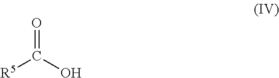
wherein
R⁵ is C2-C52 alkyl group, C2-C52 heteroalkyl group, C2-C52 alkene group, C2-C52 heteroalkene group, C3-C52 cyclic group, or C2-C52 heterocyclic group; and
the one or more mono-alcohol of (iv) is a mono-alcohol of formula (V) 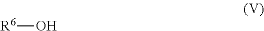
wherein
R⁶ is C2-C52 alkyl group, C2-C52 heteroalkyl group, C2-C52 alkene group, C2-C52 heteroalkene group, C3-C52 cyclic group, or C2-C52 heterocyclic group.
